(19) United States
(12) Patent Application Publication
(10) Pub. No.: US 2010/0292095 A1
(43) Pub. Date: Nov. 18, 2010

(54) HUMAN MONOCLONAL ANTIBODIES DIRECTED TO SIALYL LEWIS C, SIALYL TN AND N GLYCOLYLNEURAMINIC ACID EPITOPES AND A METHOD OF ANALYSIS OF STEM CELLS COMPRISING SAID EPITOPES

(75) Inventors: Marja-Leena Laukkanen, Espoo (FI); Kristiina Takkinen, Espoo (FI); Jari Natunen, Vantaa (FI); Tero Satomaa, Helsinki (FI); Jaakko Parkkinen, Espoo (FI); Leena Valmu, Helsinki (FI); Sari Tiitinen, Vantaa (FI); Suvi Natunen, Vantaa (FI)

Correspondence Address: BROOKS KUSHMAN P.C., 1000 TOWN CENTER, TWENTY-SECOND FLOOR, SOUTHFIELD, MI, 48075, US

(73) Assignees: SUOMEN PUNAINEN RISTI, VERIPALVELU, Helsinki (FI); GLYKOS FINLAND OY, Helsinki (FI)

(21) Appl. No.: 12/741,987

(22) PCT Filed: Nov. 10, 2008
(86) PCT No.: PCT/FI2008/050643

§ 371 Date: Jun. 21, 2010

(30) Foreign Application Priority Data
Nov. 9, 2007 (FI) 20070853

(51) Int. Cl. C40B 30/04; C07K 16/00; G01N 33/53; C07H 21/04

(52) U.S. Cl. 506/9; 530/387.9; 435/7.21; 536/23.53

(57) ABSTRACT

This invention relates to antibody engineering technology. More particularly, the present invention relates to human IgM antibodies and derivatives thereof, which have novel binding specificity with regard to several oligosaccharide sequences and/or xenoantigenic sialic acid residue. The present invention also relates to processes for making and engineering such novel saccharide and/or NeuGc-binding monoclonal antibodies and to methods for using these antibodies and derivatives thereof in the field of immunodiagnostics, enabling qualitative and quantitative determination of xenoantigenic NeuGc in biological and raw material samples, as well as in immunotherapy, enabling blocking of xenoantigenic NeuGc in patients.

### FIELD OF THE INVENTION

This invention relates to antibody engineering technology. More particularly, the present invention relates to human glycan-binding antibodies and derivatives thereof, which bind specific oligosaccharide sequences including antigenic non-human glycans The present invention also relates to processes for making and engineering such glycan-binding monoclonal antibodies and to methods for using these antibodies and derivatives thereof in the field of immunodiagnostics, enabling qualitative and quantitative determination of specific oligosaccharide sequences including antigenic non-human glycans in biological and raw material samples, as well as in immunotherapy, enabling blocking of antigenic glycans in patients, e.g., in context of a transplantation.

### BACKGROUND OF THE INVENTION

The binding specificities of human natural antibodies are not well-known. It is realized that natural antibodies may be involved in protection against malignant condition such as cancer or even autoimmune conditions or pathogenic materials such as xenoantigenic glycans. It is realized that the specificities of the natural antibodies are useful of characterization of pathogenic condition caused their production. Furthermore novel antibodies and specificities are useful for the production and optimization of novel reagents.

Table 2 lists certain antibody type protein sequences, which may be related with parts of peptide sequences of heavy chain of 1.4.30, which may be involved in carbohydrate recognition, the most preferred target oligosaccharide sequences according to the invention have not been indicated. It is realized that the publications would not indicate the whole heavy chain 1.4.30 sequence nor the novel light chain sequences. It is further realized that the carbohydrate sequences have not been indicated to the other novel antibodies such as 1.4.24 or 1.4.11 or antibodies homologous to these.

Various cell based therapies are under development. Contamination of therapeutic cells with antigenic glycan and/or xenoantigenic materials has been recognized as major problem in the development of novel cell therapies. The NeuGc has been known as a xenoantigen and an obstacle preventing xenotransplantation of organs for example from pig to human (WO02088351, Zhu, Alex), xenotransplantation is also under development by multiple biotech companies.

Antibodies against NeuGc have been published. The most published antibodies have been produced in chicken, which also lacks NeuGc-glycans like human. A method has been published for production of NeuGc-recognizing antibodies by affinity purification of chicken antibodies in column containing oxidized NeuGc lacking characteristic glycerol-radical side chain of sialic acids (Varki A et al. WO 2005010485). This method appears to be useful for purification of certain chicken antibodies. The present invention is directed to production of human natural monoclonal antibodies including the binding activity towards the glycerol part of NeuGc. It is realized that monoclonal antibodies have benefit as reagents which can be characterized and produced reproducibly by regular biotechnical method.

Two clones of human IgM antibodies produced from melanoma patients binding specifically certain oligosaccharide glycolipids has been also reported (Furukawa, K. et al., 1988). These antibodies did not recognize normal human cells or tissues nor cancer samples. The other antibody 32-27M is specific for internal NeuGc in a glycolipid not for non-reducing end terminal NeuGc in Neu5xα8Neu5Gcα3(GalNAcβ4)Galβ4Glc-tyep sequence. It did bind glycolipids in melanoma cells grown in fetal bovine serum, with possible glycolipid contamination. The other antibody recognized terminal NeuGc on certain glycolipids but no human cells under any conditions (Furukawa, K. et al., 1988). The antibodies according to the present invention were revealed to recognize specific acid saccharide epitopes and NeuGc comprising a monosaccharide and oligosaccharide structures and such structures also on human cells an proteins In context of cancer certain poorly characterized likely polyclonal NeuGc antibodies (so called Deicher-Hanganutziu antibodies) specific for the oligosaccharide glycolipid structure NeuGcα3LacβCer (GM3) has been reported. These studies appear not to represent pure human antibodies useful for analytic or therapeutic uses.

Neu5Gc recognizing P3 antibody binding specifically to NeuGc comprising GM3 ganglioside NeuGcα3Galβ4GlcβCer or sialyl-type 2 N-acetyllactosamine glycolipid NeuGcα3Galβ4GlcNAcβGalβ4GlcβCer has been known as natural mouse IgM antibody and as humanized antibody (WO9920656 Vasquez et al.). The antibody has been indicated as glycolipid specific. The present specificity excludes the type NeuGcα3Galβ4Glc(NAc)—wherein there is β4-linkage in N-acetyllactosamine together with α3-linkage for the sialic acid comprising sequences, further more P3 antibody has been reported exclusively NeuGc specific while present antibodies have sequence specific preferences for sialic acids.

The present invention reveals novel human antibodies with different peptide sequences on heavy chain and light chain with different specificities recognizing α3-sialylated type 1 N-acetyllactosamine SAα3Galβ3GlcNAc and α6-sialylated type 2 N-acetyllactosamine SAα6Galβ4GlcNAc, with both Neu5Gc and Neu5Ac. The unusual binding specificity further includes terminal sequence Neu5Acα6GalNAc, in a preferred embodiment in alfa-linked form as sialyl-Tn structure. It is realized that the present antibodies recognize preferred glycan structures on proteins and/or on proteins and glycolipids and that the specificity does not require lipid structures in the target molecules. The α3-sialylated type 2 N-acetyllactosamines and lactoses SAα3Galβ4Glc(NAc)ₙ have very low binding affinity to the present antibodies or are not recognized at all, indicating difference to the P3 type or GM3 specific antibodies.

The novel oligosaccharide sequence binding specificity is very different from the mostly ganglioside specificities in the background, including e.g. ones associated to sequences related to 1.4.30, especially heavy chain CDR1 and CDR2 regions, more specifically FTFSSYAMS type sequences. The heavy CDR1 region has certain homology to P3 and 14F7 antibodies with totally different oligosaccharide binding specificities. It is further realized that the light chain and heavy chain sequences provided by the present invention allow design and optimization of human antibodies having oligosaccharide binding activity/ies according to the invention. The human antibodies are useful for immunodiagnostics and analysis or therapies in vivo and in vitro because they are not antigenic.

It is especially realized that the present combination of α3-linked type 1 N-acetyllactosamine SAα3Galβ3GlcNAcβ and the α6-sialylated structures including O-glycan sequence SAα6GalNAcα, and even type II N-acetyllactosamine SAα6Galβ4GlcNAcβ, but practically excluding other sialyl-trisaccharide sequences is very unusual and implies to unusual two separate sialic acid binding sites in the antibodies.

The structures of the antibodies to recognize the two sequence types with either α3-linked sialic acid on secondary hydroxyl, but not related type II lactosamines, and α6-linked sialic acid on more flexible primary hydroxylstructure. It was further revealed that the specificities may not include strong recognition of at least one of the sequences SAα6Galβ4GlcNAcβ on biantennary N-glycan core structure, [SAα6Galβ4GlcNAcβ2Manα3(SAα6Galβ4GlcNAcβ2Manα6)Manβ4GlcNAcβ4GlcNAc] and it is further known that sialyl-Tn structure can not be present on N-glycans. This specificity is clearly different from preferred stem cell contamination N-glycan structures in earlier patenting of the applicants.

Several oligosaccharide sequences are known for characterization of human stem cells. The present invention is directed to unusual binder reagent recognizing several different sequences from the surface of intact cells. The recognition may involve large cell populations, an example showing almost 80% labelling of stem cells, see FIG. 9. In a parallel experiment over 80% labelling was obtained, when cells were 2 hours after detachment. The invention revealed the method especially useful for characterization of mesenchymal stem cells, especially preferred human blood related stem cells and in context of certain types of exogenous reagents and cell culture conditions or lack thereof.

It is realized that human natural antibodies are more preferred for human applications than several known antibodies from animals with potential for harmful anti-antibody immune reactions and are more likely to recognize relevant structures from human glycans.

NeuGc binding antibodies distinctively recognise xenoantigenic epitopes, which would be useful in clinics or immunodiagnostics for detecting and determining immune reactions against such materials. Production of monoclonal antibodies capable of specific binding of NeuGc-epitopes by conventional methodology such as hybridoma technology has been hampered by the presence of the structure as normal glycosylation in mice and most other animals. Phage display technology has been applied in production of antibodies against certain human complex oligosaccharide structures, wherein the effective antigenic determinant covers several monosaccharide residues. However, no data exist about phage display or other human antibodies capable of effectively recognizing a single terminal monosaccharide with only minor variation of one proton substituted by a hydroxyl group such as in antibodies binding to NeuGc-glycans but not to NeuAc-glycans. The present antibodies recognize effectively polyvalent high density conjugate of NeuGc-monosaccharide and other saccharides. The antibodies were also shown to be useful for recognition of proteins and cells including human cells. This methodology is giving new tools to produce acid oligosaccharide and/or NeuGc-specific recombinant antibodies that can be produced in consistent quality for clinical and diagnostic applications.

### SUMMARY OF THE INVENTION

We describe in this application the development and characterisation of human immunoglobulin, preferably IgM, antibody fragments that bind specifically certain novel acidic oligosaccharides including α3-sialylated type 1 lactosamines, SAα6Gal/GalNAc-structures, SAα6Galβ4GlcNAc and sialyl-Tn SAα6GalNAc and certain monosaccharide epitopess including xenoantigenic NeuGc-saccharides or corresponding NeuGc-glycans, when the antibodies have affinity and specificity high enough to be utilised as reagents in immunoassays designed for the qualitative and quantitative measurement of the saccharides and NeuGc saccharides in biological samples and, in immunotherapy e.g. in context of transplantation. Specifically, the present invention describes selection of human antibodies specific to the saccharides and/or NeuGc by an antibody library method such as the phage display technique, and the characterisation of the binding properties of the engineered antibody fragments produced in E. coli.

This invention thus provides new reagents to be utilised in different kinds of immunoassay protocols, as well as human immunotherapy. The invention also permits guaranteed continuous supply of these specific reagents of uniform quality, eliminating inherent batch-to-batch variation of polyclonal antisera. These advantageous effects permit the manufacture of new, specific and economical immunodiagnostic assays and therapeutic molecules of uniform quality.

Consequently, one specific object of the present invention is to provide human monoclonal antibodies binding saccharides according to the invention, fragments thereof, chemical or non-covalent conjugates thereof, or other derivatives of such antibodies, which bind the acidic saccharides and/or in a preferred embodiment NeuGc-glycans with affinity and specificity which allow qualitative and/or quantitative measurement of the saccharides and/or NeuGc in biological samples, as well as their use in immunotherapy. The monovalent and especially oligovalent antibodies of the present invention demonstrate a specific binding to the saccharides including xenoantigenic NeuGc-saccharides.

Another object of the present invention is to provide cDNA clones encoding specific oligosaccharide and/or NeuGc-saccharide specific antibody chains, as well as constructs and methods for expression of such clones to produce specific oligosaccharide and/or NeuGc-saccharide binding antibodies, fragments thereof or other derivatives of such antibodies. The invention is further directed to the use of the nucleic acid sequences and the complementary nucleic acid sequences and homologues thereof with the similar capacity to bind and hybridize with the nucleic acid sequences a) for analysis of expression of the nucleic acid sequences b) for effecting the expression of the nucleic acid sequences.

A further object of this invention is to provide methods of using such specific saccharide and in preferred embodiment especially NeuGc-comprising saccharide binding antibodies, fragments thereof or other derivatives of such antibodies, or combinations of them for qualitative and quantitative measurement of specific saccharide and/or NeuGc saccharide in biological samples. Additionally, this invention provides specific saccharide and/or NeuGc-binding antibodies, fragments thereof or other derivatives of such antibodies, or combinations of them for immunotherapy in patients.

Other objects, features and advantages of the present invention will be become apparent from the following drawings and detailed description. It should be understood, however, that the detailed description and the specific examples, while indicating preferred embodiments of the invention, are given for illustration only, since various changes and modifications within the spirit and scope of the invention will become apparent to those skilled in the art from this detailed description.

### BRIEF DESCRIPTION OF THE DRAWINGS

The figures of the constructions are not in scale.

FIG. 1 shows a schematic presentation of an intact human immunoglobulin antibody, Fab fragment and single-chain antibody (scFv). The domain structure and valency of the antibody depends on the selected antibody class, for example IgM comprises a pentamer of divalent antibody structures. The antigen-binding site is indicated by a triangle.

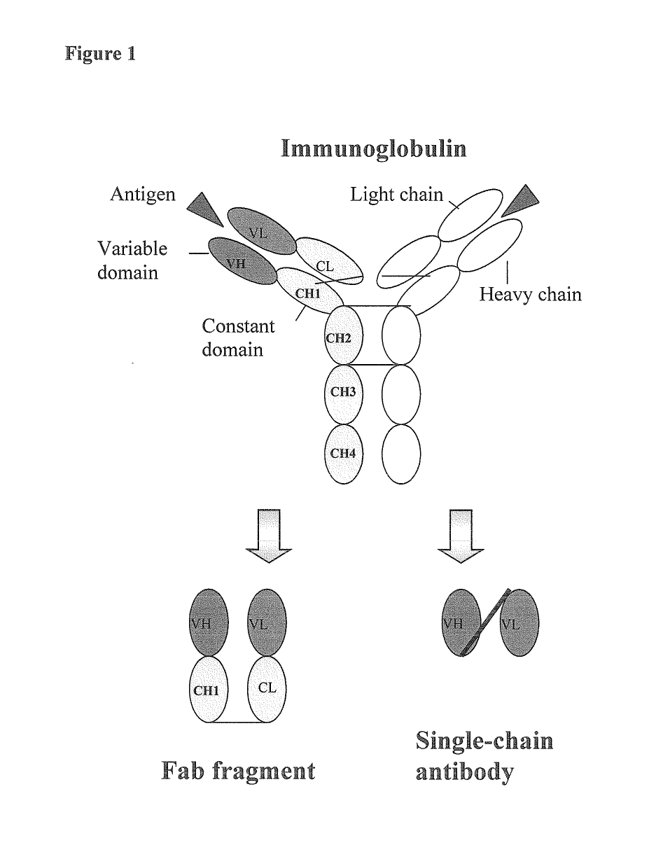

FIG. 2 shows schematically the panning procedure.

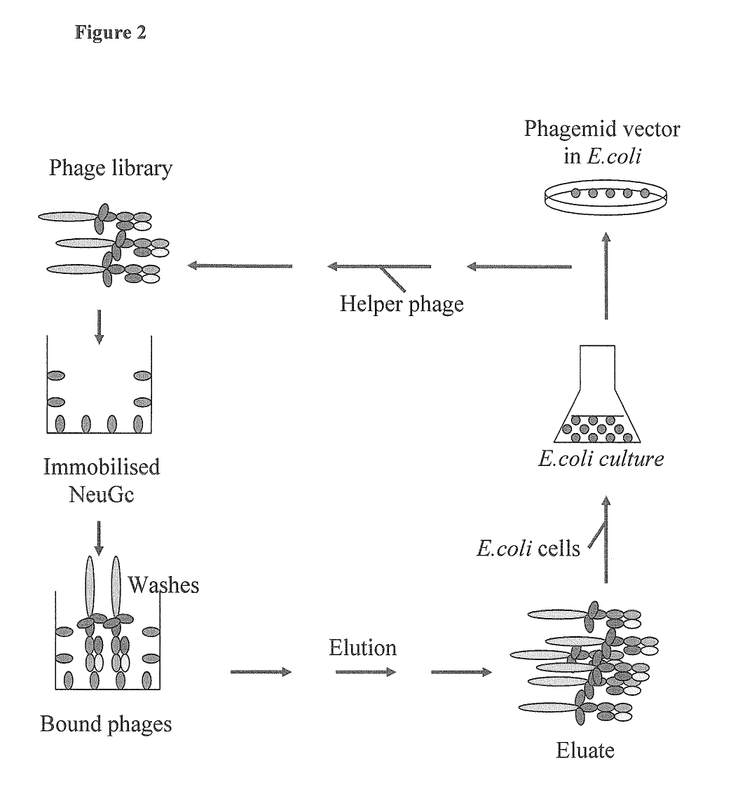

FIG. 3. The alignment of the deduced amino acid sequences of the VL region. The Complementarity Determining Regions (CDRs) are boxed. Numbering is according to Kabat (Kabat et al., 1991).

FIG. 4. The Alignment of the deduced amino acid sequences of the VH region. The Complementarity Determining Regions (CDRs) are boxed. Numbering is according to Kabat (Kabat et al., 1991).

FIG. 5. The cDNA of different VL regions. The cDNA of the VL regions were isolated by phage display technology.

FIG. 6. The cDNA of different VH regions. The cDNA of the VH regions were isolated by phage display technology.

FIG. 7. Homology of VH and VL regions at protein level. The amino acid sequence alignments in FIGS. 3 and 4 were used for drawing the protein sequence tree.

FIG. 8. Specificity of the 1.4.24 and 1.4.30 antibodies determined by immunoassay as described in experimental procedures. Both antibodies 1.4.24 and 1.4.30 are highly specific for NeuGc-monosaccharide (GF309) over naturally occurring NeuAc-monosaccharide (GF308) and also for certain other terminal saccharide epitopes according to the invention.

FIG. 9. Labelling of human cord blood mesenchymal stem cells, human CB-MSC cells, by 1.4.24 antibody in FACS (“suora leimaus”=direct labelling). The data shows a major population of intact cells labelled by the antibody, and the labelling depend on cell culture conditions.

FIG. 10. Specificity of the 1.4.19-3 (F3) antibody determined by immunoassay as described in experimental procedures. The letter A at the reducing end of the saccharides, and a in the linkage structures, e.g. (a2,6) means (alfa2,6) alfa-linkage, and letter B at the reducing end of the saccharides, and b in the linkage structures mean beta anomeric structures. Polyvalent polyacrylamide saccharide conjugates were from Lectinity Holdings Russia or were synthesized by sialyltransferase reactions (enzymes from Calbiochem) from these using CMP-Neu5Ac or CMP-Neu5Ge as donors and structures of glycans were verified by NMR spectroscopy.

FIG. 11a. The cDNA and protein sequences of scFv 1.4.19-3 (F3).

FIG. 11b. The heavy and light chain CDR regions of scFv 1.4.19-3 (F3) clone, with heavy chain structure corresponding to 1.4.24 and light chain structure corresponding to 1.4.30.

### ABBREVIATIONS

cDNA complementary deoxyribonucleic acid
CDR complementarity determining region
DNA deoxyribonucleic acid
E. coli Escherichia coli
ELISA enzyme-linked immunosorbent assay
Fab fragment with specific antigen binding
Fd variable and first constant domain of a heavy chain
Fv variable regions of an antibody with specific antigen binding
GFP green fluorescent protein
IgM immunoglobulin M
mRNA messenger ribonucleic acid
NeuAc Neu5Ac, N-acetylneuraminic acid
NeuGc Neu5Gc, N-glycolylneuraminic acid
NMR nuclear magnetic resonance
PCR polymerase chain reaction
RNA ribonucleic acid
scFv single-chain antibody
SA, Sialic acid, Neuramic acids including NeuGc and NeuAc
supE⁻ a genotype of bacterial strain carrying a glutamine-inserting amber suppressor tRNA
V_{H} variable region of a heavy chain
V_{L} variable region of a light chain

### DETAILED DESCRIPTION OF THE INVENTION

Applicants have other co-pending inventions about glycan marker structures of stem cells e.g. WO/2007/006864, WO 2008/107522, WO/2008/087259, WO/2008/087258, WO/2008/087257, WO/2007/006870, included fully as reference.

Binder molecules/reagents bind glycans and preferably include property allowing observation of the binding such as a label linked to the binder. The novel glycan specificity against the rigid glycan structures define structurally the conformation of reagents binding to the glycans, structures are available e.g. from internet pages of sweetdb, www.glycosciences.de/sweetdb/index php. The preferred binders include a) Proteins such as antibodies, lectins and enzymes b) Peptides such as binding domains and sites of proteins, and synthetic library derived analogs such as phage display peptides c) Other polymers or organic scaffold molecules mimicking the peptide materials including aptamers and the like.

The peptides and proteins are preferably recombinant proteins or corresponding carbohydrate recognition domains derived thereof, when the proteins are selected from the group monoclonal antibody, glycosidase, glycosyl transferring enzyme, plant lectin, animal lectin or a peptide mimetic thereof, and wherein the binder includes a detectable label structure, it is realized that based on sequence data and molecular modelling it is possible to design binder molecules like the present antibodies. Antibodies and fragments there of are most preferred binder reagents.

The following definitions are provided for some terms used in this specification. The terms, “immunoglobulin”, “heavy chain”, “light chain” and “Fab” are used in the same way as in the European Patent Application No. 0125023.

“Antibody” in its various grammatical forms is used herein as a collective noun that refers to a population of immunoglobulin molecules and/or immunologically active portions of immunoglobulin molecules, i.e., molecules that contain an antigen binding site or a paratope. Examples of molecules which are described by the term “antibody” herein include, but are not limited to: single chain Fvs (sdFvs), Fab fragments, Fab′ fragments, F(ab′) fragments, disulfide linked Fvs (sdFvs), Fvs, and fragments comprising or alternatively consisting of, either a VL or a VH domain. The immunoglobulin molecules of the invention can be of any type (e.g., IgG, IgE, IgM, IgD, IgA and IgY), or subclass of immunoglobulin molecule. Preferably, an antibody of the invention comprises, or alternatively consists of, a VH domain, VH CDR, VL domain, or VL CDR.

An “antigen-binding site”, a “paratope”, is the structural portion of an antibody molecule that specifically binds an antigen.

Exemplary antibodies are those portions of an immunoglobulin molecule that contain the paratope, including those portions known as Fab and Fv.

“Fab” (fragment with specific antigen binding), a portion of antibody can be prepared by the proteolytic reaction of papain on substantially intact antibodies by methods that are well known. See for example, U.S. Pat. No. 4,342,566. Fab fragments can also be produced by recombinant methods, which are well known to those skilled in the art. See, for example, U.S. Pat. No. 4,949,778.

“Domain” is used to describe an independently folding part of a protein. General structural definitions for domain borders in natural proteins are given in Argos, 1988.

A “variable domain” or “Fv” is used to describe those regions of the immunoglobulin molecule, which are responsible for antigen or hapten binding. Usually these consist of approximately the first 100 amino acids of the N-termini of the light and the heavy chain of the immunoglobulin molecule.

“Single-chain antibody” (scFv) is used to define a molecule in which the variable domains of the heavy and light chain of an antibody are joined together via a linker peptide to form a continuous amino acid chain synthesised from a single mRNA molecule (transcript).

“Linker” or “linker peptide” is used to describe an amino acid sequence that extends between adjacent domains in a natural or engineered protein.

A “NeuGc-binding antibody” is an antibody, which specifically recognises NeuGc and binds to it, due to interaction mediated by its variable domains. Specific recognition means higher binding activity towards specific saccharide in comparison to the corresponding control saccharide.

“Saccharide” means monosaccharide or oligosaccharide epitope. The saccharide epitopes are preferably non-reducing end terminal saccharides, which may be elongated preferably only from its reducing end. The elongation may be to a larger carbohydrate structure and/or elongation by linkage to a carrier such as a protein: a polymer including polyacrylamides, polypeptides, dendrimers or polysaccharides; or a lipid comprising a hydrophobic aglycon. The preferred polymer structures further include natural and/or non-natural carbohydrate structures e.g. in synthetic neoglycoproteins or neoglycolipids or saccharide polymer conjugates comprising a linkage to monovalent aglycon structure or spacer to a carrier structure such as a polymer.

In context of analysis of biological materials, such as antibodies according to the invention or sera or libraries comprising these, preferred oligosaccharide epitopes include non-reducing end terminal oligosaccharide sequences, more preferably elongated oligosaccharide sequences, and natural the carrier structures are preferably natural glycoconjugates such as protein(s), including O-glycan and/or N-glycan structures linked to proteins and/or lipid structures such as glycosphingo lipids comprising a ceramide at the reducing end. The epitopes may be in preferred embodiment part of polysaccharide such as branched bacterial polysaccharide, known and modifiable in prior art e.g. as described part of the inventors. More preferably the saccharides are elongated solely form the reducing end furthermore the oligosaccharide sequences are preferably not modified or derived by any additional groups to any hydroxylgroup structure, which is not the reducing end. The elongating structure may be a natural sequence of the natural glycan recognized such as O-glycan, N-glycan or glycolipid (preferably glycospingo lipid) structure.

The single monosaccharide residues are linked by alfa- or beta-glycosidic linkage to a non-monosaccharide material such as spacer structures, preferably glycosidically linked alkyl spacer, linking glycans to polymers, in a preferred embodiment to polypeptides, dendrimers or polyacrylamides, more preferably polyacrylamides. The invention revealed binding to both alfa- and beta linked sialic acids, preferably Neu5Gc and glucuronic acid, preferably GlcA with alfa or beta linkage. The invention is further directed to the analysis of binding to GlcA in natural glycans comprising GlcA, especially glycosaminoglycans and/or glyco lipids. The invention is further directed to the analysis of antibody binding to uronic acid containing monosaccharide residues in oligosaccharide sequences in the middle polysaccharide sequences such as in glycosaminoglycans.

Glyco lipid and carbohydrate nomenclature is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 29).

It is assumed that Gal (galactose), Glc (glucose), GlcNAc (N-acetylglucosamine), GalNAc (N-acetylgalactosamine) and Neu5Ac are of the D-configuration, Fuc of the L-configuration, and all the monosaccharide units in the pyranose form. The amine group is as defined for natural galactos- and glucosamines on the 2-position of GalNAc or GlcNAc. Glycosidic linkages are shown partly in shorter and partly in longer nomenclature, the linkages of the sialic acid SA/NeuSX-residues α3 and α6 mean the same as α2-3 and α2-6, respectively, and with other monosaccharide residues α1-3, β1-3, β1-4, and β1-6 can be shortened as α3, β3, β4, and 136, respectively. Lactosamine refers to type II N-acetyllactosamine, Galβ4GlcNAc, and/or type 1 N-acetyllactosamine, Galβ3GlcNAc and sialic acid (SA) is N-acetylneuraminic acid (Neu5Ac) or N-glycolylneuraminic acid (Neu5Gc) or any other natural sialic acid including derivatives of NeuSX. The sialic acid are referred together as NeuNX or NeuSX, wherein preferably X is Ac or Gc. Occasionally Neu5Ac/Gc/X may be referred as NeuNAc/NeuAc/NeuNGc/NeuGc/NeuNX. Term glycan means here broadly oligosaccharide or polysaccharide chains present in human or animal glycoconjugates, especially on glyco lipids or glycoproteins.

Glycan epitope or epitopes mean oligosaccharide sequence and elongated epitope means reducing end elongated preferred oligosaccharide sequence variants.

“Oligosaccharide sequence” means specific sequence of glycosidically linked monosaccharide residues, preferably including terminal and “core” sequences, The core oligosaccharide sequences can be modified by non-reducing end monosaccharide residue(s). The expression “terminal oligosaccharide sequence” indicates that the oligosaccharide is not substituted to the non-reducing end terminal residue by another monosaccharide residue or residues. Preferably the non-reducing end of the oligosaccharide sequence consists of the oligosaccharide sequence and it is only modified from the reducing end of the oligosaccharide sequence, preferably it is glycosidically conjugated from the reducing end.

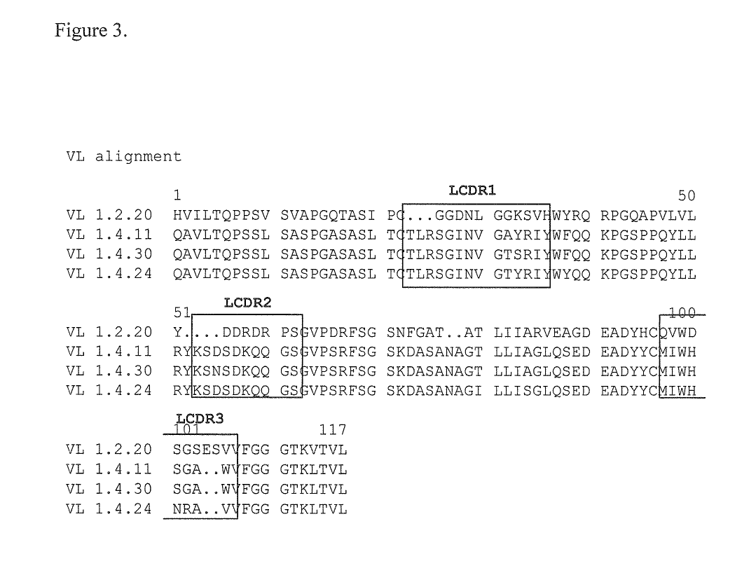

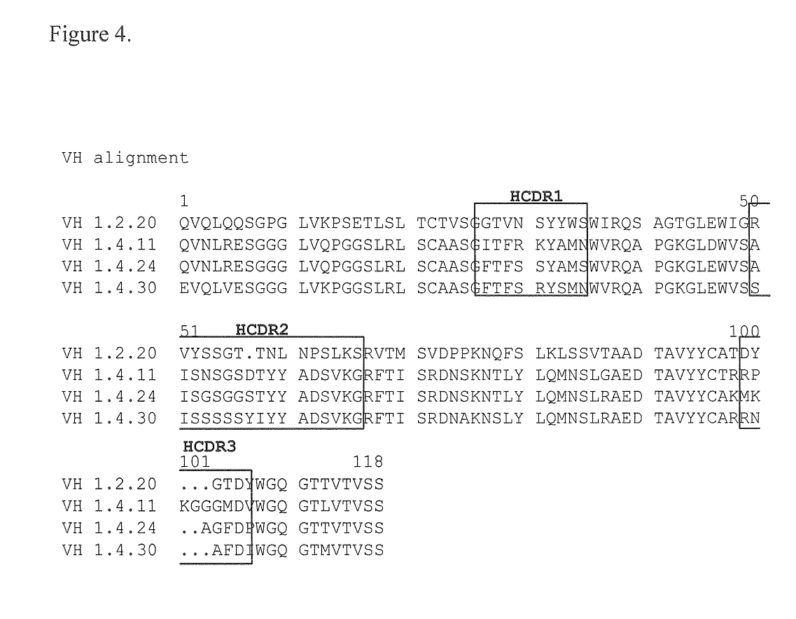

As examples of fragments of such antibodies falling within the scope of the invention we disclose here scFv fragments as shown in FIGS. 3 and 4. In one preferred embodiment, the present invention thus provides derivatives of NeuGc and/or saccharide-binding antibodies, e.g. Fab fragments or scFv fragments. It will be appreciated that mutant versions of the CDR sequences or complete V_{L} and V_{H} sequences having one or more conservative substitutions which do not substantially affect binding capability, may alternatively be employed.

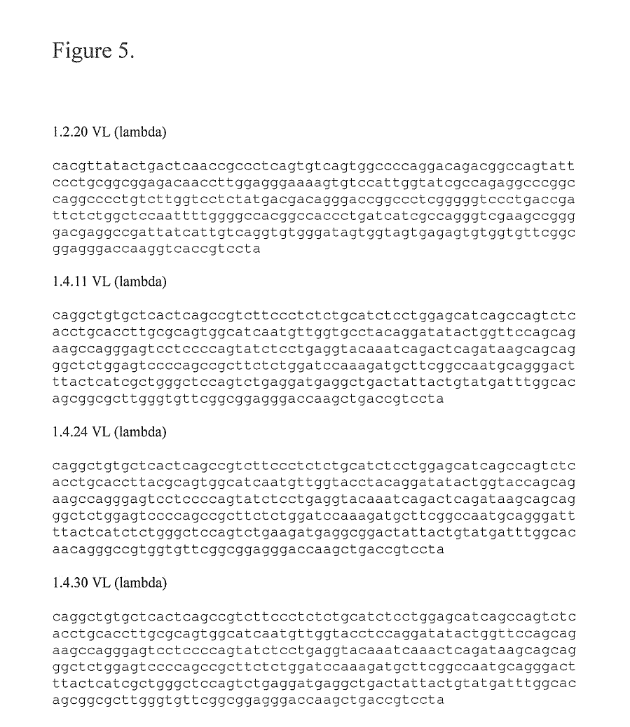

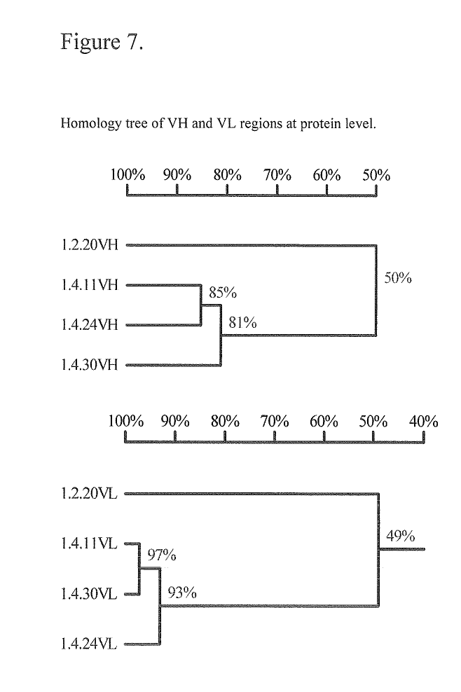

The novel antibody sequences were reproducibly produced from large pool on IgM genes from about 50 persons. The invention revealed that the antibody sequences share substantial homology as shown for 4 antibodies in examples and in figures. It is realized that each antibody sequence is valuable as such natural type human antibody sequence recognizing the important antigen. The present invention is directed to antibodies having substantial homology or similarity with sequences of light chain (VL) and/or of heavy chain (VH). It is realized that the sequence homologies are substantial on both protein and nucleic acid, such as cDNA-level. In a preferred embodiment the peptide (protein) sequences of the antibody domains are compared. The present invention is directed to antibodies having substantial homology with sequences of light chain (VL) sequences in FIG. 3 (or corresponding DNA in FIG. 5, realizing that the exact homology % vary from the one defined for proteins), as shown in FIG. 7, all antibodies share protein level homology of about 50%, more specifically at least about 49%, three protein sequences 1.4.11, 1.4.24, and 1.4.30, referred as 1.4-group share even higher homology of at least about 90%, more precisely at least 93% for the specific sequences. The analysis further reveals a preferred subgroup of 1.4.11 and 1.4.30 type antibodies even sharing about 95%, more precisely about 97% homology with each other, the homology being close to identity of the sequences.

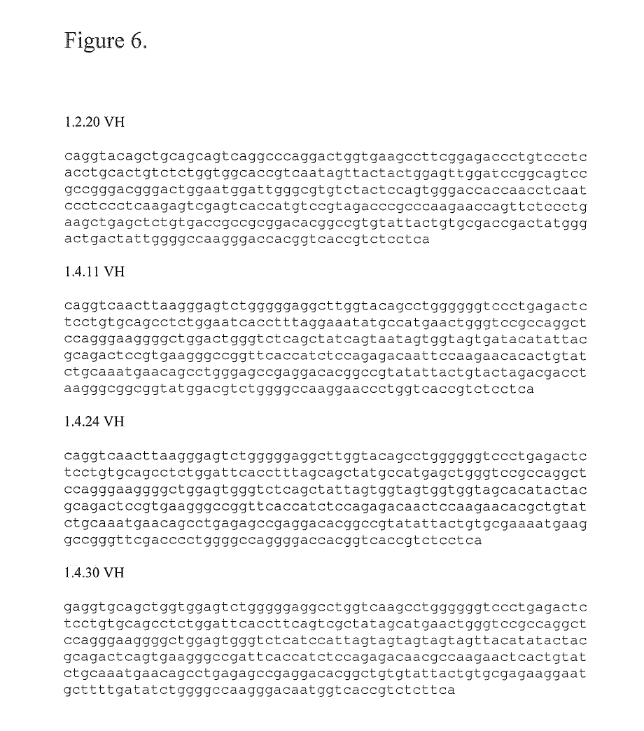

The present invention is directed to antibodies having substantial sequence homology with sequences of heavy chain (VH) sequences in FIG. 4 (or corresponding DNA in FIG. 6, realizing that the exact homology % vary from the one defined for proteins), as shown in FIG. 7, all antibodies share protein level homology of about 50%, three protein sequences 1.4.11, 1.4.24, and 1.4.30, referred as 1.4-group share even higher homology of at least about 80%, more precisely at least 81% for the specific sequences. The analysis further reveals a preferred subgroup of 1.4.11 and 1.4.24 type antibodies even sharing about 85% homology with each other.

Defining Consensus Sequences for Specific Saccharide and/or NeuGc Antibodies

The present invention is directed to methods of defining consensus sequences for specific saccharide recognizing and/or NeuGc antibodies by comparing the antibody sequences according to the invention and optionally other antibodies. The present invention is further directed to methods of defining unusual characteristic sequences for specific saccharide and/or NeuGc antibodies or antibody groups by comparing the antibody sequences according to the invention and optionally other antibodies. The present invention is especially directed to comparison of CDR-sequences, as shown for example in boxes in FIGS. 3 and 4 for both light and heavy chains, as CDRs known to be essential for the binding properties of antibodies.

### Light Chain Consensus Sequences

The invention is in a preferred embodiment directed to the following consensus sequences for light chains of 1.4 group antibodies:

CDR1: TLRSGINVGX₁X₂RIY, wherein X₁ is preferably A or T and X₂ is Y or S
CDR2: KS X₁SDKQQGS, wherein X₁ is preferably N or D.
CDR3: MIWHX₁X₂AX₃WV, wherein X₁ is preferably S or N and X₂ is G or R and X₃ is W or V.

It is noticed that the homology is high within the CDR-sequences. An antibody of 1.4 group comprise all the characteristic light chain which preferably has CDRs similar or essentially similar to the CDR1-3 sequences.

The invention is in a preferred embodiment directed to following consensus sequences for light chains of 1.2.20 type antibodies:

CDR1: GGDNLGGKSVH,
CDR2: DDRDRPS,
CDR3: QVWDSGSESVV,

An antibody of 1.2.20 type comprise the characteristic light chain, which preferably has CDRs similar or essentially similar to the CDR1-3 sequences.

There are characteristic differences between the 1.4 group and 1.2.20 type antibodies such as the lengths of the light chain CDRs, while the first two CDRs are shorter in 1.2.20 antibodies, the CDR3 is longer for the 1.2.20. However common motives can be find for both types of the antibodies:

CDR1, residues 27-45: GX₁NZ₁GX₂X₃X₄Z₂, wherein X₁ is preferably D or I and X₂ is G, A, or T, and X₃ is K, Y, or S; and X₄ is S, or R; and Z₁ and Z₂ are aliphatic chain comprising hydrophobic amino acid residues, preferably Z₁ is L, or V; and Z₂ is V or I.
CDR2, residues 57-62: DZ₁X₁X₂X₃S, wherein X₁ is preferably D or Q; and X₂ is R, or Q; and X₃ is P, or G; and Z₁ is basic chain comprising polar amino acid residue, preferably Z₁ is R, or K.
CDR3, 98-102: Z₁WX₁X₂X₃, wherein X₁ is preferably D or H; and X₂ is S, or N; and X₃ is G, or R; and Z₁ is an aliphatic chain comprising hydrophopic amino acid residues, preferably Z₁ is V or I.

Preferred CDR3 sequences further include Z₁WX₁SG, wherein X₁ is preferably D or H; and Z₁ is V or I. This sequence is preferred common sequence for 1.2.20, 1.4.11 and 1.4.30.

### Heavy Chain Consensus Sequences

The invention is in a preferred embodiment directed to the following consensus sequences for heavy chains of 1.4 group antibodies:

CDR1: X₁TFX₂X₃YX₄MX₅, wherein X₁ is preferably I or F; and X₂ is R or S; and X₃ is K, or S, or R; and X₄ is A or S; and X₅ is N or S.
CDR2: X₁₁SX₂SX₃X₄X₅X₆YYADSVKG, wherein X₁ is preferably A or S; and X₂ is N, G, or S; and X₃ is G, or S; and X₄ is S or G; and X₅ is D, S or Y; and X₆ is T or I.
CDR3: X₁X₂X₃X₄X₅X₆X₇DX₈, wherein X₁ is preferably R or M: and X₂ is P, K or N and X₃ is K or nothing; and X₄ is G or nothing; and X₅ is G, A, or nothing; and X₆ is G, or A; and X₇ is M, or F, and X₈ is V, or P or I.

It is noticed that the homology is high within the CDR-sequences. An antibody of 1.4 group comprise all the characteristic heavy chain which preferably has CDRs similar or essentially similar to the preferably CDR1 and CDR2 and most preferably all CDR1-3 sequences.

The invention is in a preferred embodiment directed to following consensus sequences for heavy chains of 1.2.20 type antibodies:

CDR1: GTVNSYYWS,
CDR2: RVYSSGTTNLNPS,
CDR3: DYGTDY

An antibody of 1.2.20 type comprise the characteristic heavy chain, which preferably has CDRs similar or essentially similar to preferably CDR1 and CDR2 and most preferably all the CDR1-3 sequences.

There are characteristic differences between the 1.4 group and 1.2.20 type antibodies such as the lengths of the heavy chain CDRs, while the second CDRs of 1.2.20 antibodies is shorter than in the others, the CDR3 is also shorter for the 1.2.20 and for the 1.4.30, too. However common motives can be found for both types of the antibodies:

CDR1, residues 27-35: X₁TZ₁X₂X₃YX₄Z₂X₅, wherein X₁ is preferably G, I or F; and X₂ is N, R or S; and X₃ is K, or S, or R; and X₄ is Y, A or S; and X₅ is N or S; and, preferably Z₁ is V, or F; and Z₂ is W or M.
CDR2: X₁Z₁Z₂X₂SX₃X₄X₅X₆Z₃Z₄Z₅Z₆SZ₇KZ₈, wherein X₁ is preferably R, A or S; and X₂ is N, G, or S; and X₃ is G, or S; and X₄ is T, S or G; and X₅ is nothing, D, S or Y; and X₆ is T or I; and, Z₁ is V, or I; and Z₂ is Y or S; and Z₃ is N or Y; and Z₄ is L or Y; and Z₅ is N or A; and Z₆ is P or A; and Z₇ is L or V; and Z₈ is S or G.
CDR3: X₁X₂X₃X₄X₅X₆X₇DX₈, wherein X₁ is preferably D, R or M: and X₂ is Y, P, K or N and X₃ is K or nothing; and X₄ is G or nothing; and X₅ is G, A, or nothing; and X₆ is G, or A; and X₇ is T, M, or F; X₈ is Y, V, or P or I.

Preferred heavy chain CDR3 sequences include the conserved D residue at second last position.

It is realized that the conserved CDR protein or nucleic acids such as DNA sequences are useful for the recognition of the antibodies or corresponding nucleic acid expression in assays such as assays by specific saccharide entigens or antibodies recognizing the protein sequences and/or by RNA/DNA analysis such as PCR analysis for recognition of the corresponding nucleic acid expression.

### Comparative Analysis of Saccharide and NeuGc-Recognizing Antibodies

The data reveals that especially the three protein sequences the 1.4-group, share large sequence homology forming a homogeneous group of antibodies with some specific characteristics for each antibody. The similarities allow analysis of conserved structures of the 1.4-group of antibodies. In a specific embodiment the invention is directed to the 1.4-group antibodies as a preferred type of saccharide and NeuGc-recognizing antibodies, and use of the antibody protein or nucleic acid sequence(s) for comparative analysis of other potentially saccharide and NeuGc-recognizing antibodies.

The sequence 1.2.20 has distinct sequence, but shares some specific sequence characteristics similar with the 1.4-group. In a specific embodiment the invention is directed to 1.2.20 type antibodies as a preferred type of saccharide and NeuGc-recognizing antibodies, and use of the antibody protein or nucleic acid sequence for comparative analysis of other potentially saccharide and NeuGc-recognizing antibodies. The similarities between the four sequences allow analysis of conserved structures of the 1.4-group of antibodies and the 1.2.20 like antibodies.

Comparison of the Antibody Groups with Known Antibodies and Antibody Sequences

The present invention is further directed to methods of comparing the antibodies according to the invention either structurally and/or functionally with known antibodies, preferably antibodies, which are likely to have similar structure and/or function. The invention is especially directed to comparison the antibodies with known NeuGc recognizing antibodies such as known polyclonal antibodies produced in chicken or known NeuGc-recognizing monoclonal antibodies such as antibodies cloned from human described by Furukawa et al. 1988, with distinct and different specificities and monoclonal antibodies produced in mice by immunization such as P3-type antibodies produced in Havana Cuba and known to recognize NeuGc-comprising glycolipids (Moreno et al., 1998; Vásquez et al., WO9920656).

The functional binding of the antibodies is preferably compared for the binding with the polyvalent conjugate used in the present invention and/or protein and/or lipid bound saccharide or NeuGc-comprising structures present on cell materials. The protein binding of present antibodies and possible comparison antibodies can be performed by any suitable protein interaction method, and is preferably performed by a solid phase assay method such as Western-blot method.

The invention is further directed to the comparison of the sequences, preferably the protein sequences, of the present antibodies with other antibody sequences, preferably from antibodies, which are likely to have similar structure and/or function. The preferred antibodies with similar function include acid carbohydrate recognizing antibodies, preferably carboxylic acid comparising carbohydrate such as GlcA or Sialic acid, and more preferably sialylated carbohydrate, and in a preferred embodiment NeuGc-carbohydrate recognizing antibodies.

It is further realized that it would be useful to compare the present antibody sequences with antibody sequences known from patients of autoimmune diseases and/or cancer, because certain types of human immune responses with potential recognition of NeuGc type structures are associated with these diseases. The invention is directed to method of comparing antibody sequences associated with these diseases, preferably human antibody sequences from cancer and/or autoimmune diseases and preferably selecting antibody sequences with homology % with regard to heavy and/or light chains, in range of preferred antibodies/antibody groups according to the invention and preferably testing such antibody/antibodies with regard to binding to NeuGc comprising carbohydrate. It is realized that such experiments are very useful for revealing causes of and designing potential treatments for the diseases.

In a preferred embodiment the antibody sequence for comparison is cloned from a person who has had a blood contact with NeuGc material, such as transplantation/injection with biological reagent or material comprising NeuGc, preferred transplantation is organ/tissue transplantation with material comprising NeuGc, preferred organ transplantation further includes stem cell transplantation with possibility of contamination with NeuGc.

Novel Antibodies with Useful Glycan Binding Specificities

The present invention revealed a library of monoclonal antibodies with novel and useful monosaccharide and oligosaccharide binding specificities. The antibodies have binding specificity profile, which is useful for the analysis of multiple cell types especially human cells.

The binding specificity includes several useful glycan types, part of which are specific for the subtypes of cells. The invention is in a preferred embodiment directed to selecting an antibody from the present antibodies for the binding of specific subtype of human cells. It is further realized that the antibodies are useful for the sorting of the cells.

### Disease Associated Antibodies

The invention further reveals that there is substantial homology in part of the heavy chains of the present antibody (/antibodies) with antibodies recognizing important antigenic structures in context of cancer and/or autoimmune diseases. The specificities of antibodies cannot be produced directly from the sequences, especially when the three dimensional structures are not known.

However, the present invention reveals that there are novel carbohydrate binding specificities among the antibodies which comprise heavy chain CDRs according to the invention, especially CDR1 and CDR2, according to the invention, especially in the antibodies homologous to 1.4.24.

A major problem of the development and analysis of these antibodies is that their potential and/or exact carbohydrate binding specificities have not been known. The present invention provides methods for revealing the carbohydrate specificities of antibodies recognizing especially acidic monosaccharide residue comprising structures such as sialic acids (Neu5Gc and Neu5Ac) and glucuronic acid comprising structures.

### Assay for Development or Analysis of an Antibody

The invention is directed to the method of analysis of disease associated or a cell binding antibody, preferably human antibody, wherein the method includes step of measuring the specificity of the antibody towards the saccharides including the monosaccharide and oligosaccharide sequences according to the invention.

Preferably the binding of the antibody is measured with regard to at least oligosaccharide sequences, and more preferably at least to two key oligosaccharide sequence, more and most preferably to three key oligosaccharide sequences according to the invention. In a further preferred embodiment the antibody binding to the control saccharides, according to the invention, including preferably at least one, more preferably at least two and most preferably at least three control oligosaccharide sequences is measured.

The preferred analysis method includes step of contacting an antibody with the preferred saccharide sequence or sequences according to the invention.

Further preferred step includes measuring the complex formed between the saccharide and the antibody. The preferred methods for observing the complex includes methods for measuring distance of molecules such as fluorescence method including FRET, and methods of removing non-bound reagent from the assay, such as washing the non-bound reagent such as the antibody and measuring the bound reagent such as the antibody by standard methods including detection methods such as enzyme, fluorescence or radiolabel based methods, the preferred enzyme linked assays includes ELISA assays. It is realized that the assay may be a solid phase assay.

### Monosaccharide Binding Specificities

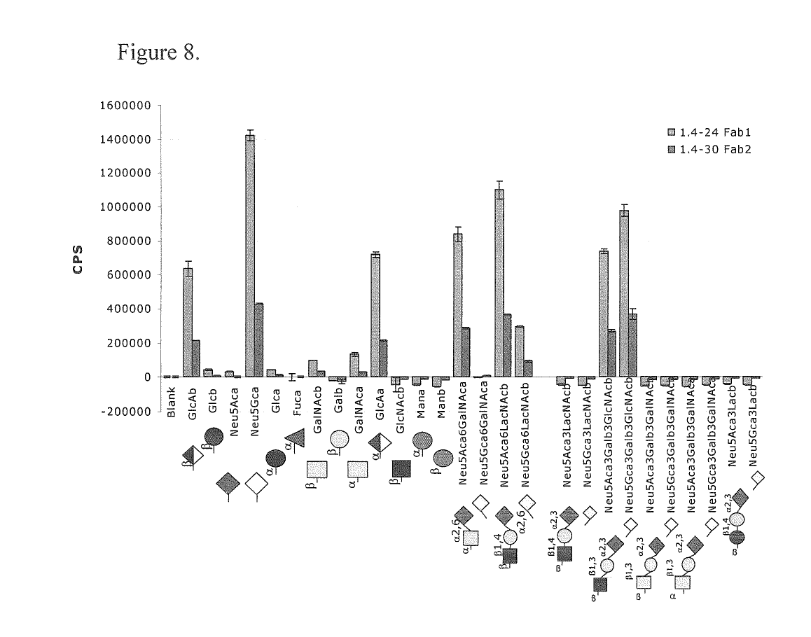

The analysis revealed that novel antibodies have affinity towards monosaccharide residues, when analysed as polyacrylamide conjugates comprising flexible spacer structures: Neu5Gcα, GlcAα, GlcAβ, GalNAcα, GalNAcβ. It is realized that recognition of these monosaccharide residues as terminal parts of oligosaccharide chains may require similar flexible representation of the structures, especially for Neu5Gc. Several neutral non-reducing terminal monosaccharide residues especially alfa- and beta linked Glc, Man, GlcNAcβ-, Galβ- and Fucα- were practically negative in the binding experiments, FIG. 8.

The best binding was to the acid monosaccharide residues glucuronic acid and Neu5Gc sialic acid. Furthermore the oligosaccharide binding specificities revealed binding to several Neu5Ac comprising oligosaccharide sequences.

The invention is in a preferred embodiment directed to the development of antibodies for the recognition of sialic acid comprising glycans and more preferably antibodies specific for Neu5Ac or Neu5Gc comprising glycans.

### Neu5Gc Comprising Glycans

In a preferred embodiment the invention is directed to novel antibodies binding more strongly (recognizing more specifically) Neu5Gc than Neu5Ac oligosaccharide sequence. In a preferred embodiment the Neu5Gc oligosaccharide sequence bound/recognized is type 1 N-acetyllactosamine sequence Neu5Gcα3Galβ3GlcNAc. In a preferred embodiment Neu5Gcα3Galβ3GlcNAc is recognized by more efficiently than Neu5Acα3Galβ3GlcNAc, preferably in an ELISA-type assay.

### Neu5Ac Comprising Glycans

In a preferred embodiment the invention is directed to novel antibodies binding more strongly (recognizing more specifically) Neu5Ac than Neu5Gc oligosaccharide sequence, more preferably the antibody binds to Neu5Ac but much weakly or practically not at all to Neu5Gc. In a preferred embodiment the Neu5Ac oligosaccharide sequence bound/recognized is sialylated non-reduging end terminal Neu5AcαGalGalNAc-structure, more preferably sialyl-Tn sequences Neu5Acα6GalNAcα. It is realized that selective recognition of Neu5Ac structure is also a useful property for an antibody, and it is in a preferred embodiment used for differentiation between human and animal glycan structures.

### Novel Oligosaccharide Binding Specificities

The invention revealed highly specific recognition of a few important key oligosaccharide sequences
a) β3-sialylated type 1 N-acetyllactosamine sequence SAα3Galβ3GlcNAc, wherein SA is Neu5Gc or Neu5Ac, more preferably Neu5Gcα3Galβ3GlcNAc, or even more preferably Neu5Gcα3Galβ3GlcNAc is recognized or bound with higher affinity than Neu5Acα3Galβ3GlcNAc.
b) α6-sialylated type 2 N-acetyllactosamine sequence SAα6Galβ4GlcNAc, wherein SA is Neu5Gc or Neu5Ac, including Neu5Acα6Galβ4GlcNAc, and Neu5Gcα6Galβ4GlcNAc. A preferred target recognized with higher affinity is Neu5Acα6Galβ4GlcNAc.
c) Sialylated non-reduging end terminal Neu5Acα6GalNAc-structures preferably sialyl-Tn sequences Neu5Acα6GalNAcα.

The invention further revealed useful control oligosaccharide sequences, with much lower or no binding to the antibodies including: α3-sialylated type II N-acetyllacotsamines and lactoses SAα3Galβ4Glc(NAc)ₙ, wherein SA is Neu5Gc or Neu5Ac and n is 0 or 1. These glycans indicate the antibodies are specific in the recognition of the glycans.

These binding specificities and combinations thereof are novel for human monoclonal antibodies, especially for natural monoclonal human antibodies. It is realized that presence of multiple but highly selective glycan recognitions by human monoclonal antibody is somewhat unusual. Furthermore it is realized that due to species specificity of glycosylation, antigenicity of a structure cannot be known from results from experiments from other species such as mice, rats or rabbits commonly used in immunizations. In case of polyclonal antisera, the actual binding specificity can not been derived from results with polyclonal antibodies and antisera.

Analysis of the Binding with Regard to Larger Structures

It is realized that the antibody binding mono- or oligosaccharide structures or epitopes thereof according to the invention, may be part of larger oligosaccharide sequences, which would be recognized with higher or lower affinity than the present oligosaccharide epitopes.

The present invention is directed to screening of other glycans, especially with larger oligosaccharide sequences present on natural glycolipids or glycoproteins, in a binding assay including step of comparing the binding of the antibody to the other saccharide with the mono- or oligosaccharide structure according to the present invention, preferably including at least one novel binding sequence according to the present invention.

Further Development by Mutagenesis and/or by Replacement of the Sequences

The recognition of both type 1 and 2 with lactosamines with specifically different α3- and α6-linked sialic acid structures and even α6-sialylated GalNAc and different binding to Neu5Gc and Neu5Ac in various constructs indicates that the antibodies recognize multiple conformations of glycans and have at least two different sialic acid binding sites or conformations.

The invention is directed to the methods of changing of the antibody specificities for development of new antibodies by changing the peptide sequences of the antibodies in the variable regions by mutagenesis methods and/or by replacing the one or more variable CDR-sequences or parts thereof from one antibody by corresponding sequence from another antibody. In a preferred embodiment the mutagenesis method is combined with the carbohydrate binding assay according to the invention and the binding of the modified antibodies to specific acid glycan structures according to the invention is measured to reveal the altered specificity.

### Novel Protein Expressed or Protein/and Lipid Expressed Target Glycans

In a preferred embodiment the present invention is directed to development and analysis of antibodies recognizing protein type glycan target sequences. The invention revealed that there is binding to sialyl-Tn sequence NeuNAcα6GalNAcα, which is present on mucin type glycoproteins. The invention is directed to human monoclonal antibodies recognizing the structure, especially antibodies comprising consensus sequences or substantial homology with present antibodies.

It is further realized that α3-sialylated type 1 N-acetyllactosamine sequence SAα3Galβ3GlcNAc and α6-sialylated type 2 N-acetyllactosamine sequence SAα6Galβ4GlcNAc can be presented both by proteins and glycolipids. The invention is directed to the use of the antibodies for analysis of the structures from protein and lipids. The invention is further directed to human monoclonal antibodies recognizing the structure, especially antibodies comprising consensus sequences or substantial homology with present antibodies.

The present invention is especially directed to novel antibodies, preferably human antibodies, and further development and assays thereof directed to these, when the antibodies recognizes Neu5Ac or Neu5Gc on protein linked glycans. In a preferred embodiment the present invention is directed to human natural antibodies binding to protein linked Neu5Gc saccharide sequences.

The invention is directed to the analysis of binding of natural human antibody to saccharide sequences according to the invention, preferably Neu5Gc comprising oligosaccharide sequences, when the saccharide sequences are linked to proteins.

The invention is directed to the analysis of binding of natural human antibody to saccharide sequences according to the invention, preferably Neu5Gc comprising oligosaccharide sequences, when the saccharide sequences are linked to lipids. The invention is especially directed to analysis of binding to lacto- and neolactoseries glyco lipids comprising the terminal epitopes according to the invention.

### Measurement of Antibody Binding in Context of Nutritional or Therapeutic Proteins

It is realized that Neu5Gc linked oligosaccharide can be present on glycoproteins which may get to contact with human in therapeutic or nutrition contexts and cause immune reactions. In a preferred embodiment the invention is directed to analysis of human antibodies against the oligosaccharide sequences according to the invention, when the oligosaccharide sequences are linked to a therapeutic protein, preferably a therepautic recombinant protein.

In a preferred embodiment the antibody to be measured in assay according to the invention has homology to the antibodies according to the invention. It is realized that homology to present amino acid sequences can be used a method step of selecting antibodies for saccharide binding analysis according to the invention. In a preferred embodiment the antibody has at least one variable region according to the concensus sequence according to the invention, or has sequence at least 70%, more preferably at least 80% and most preferably at least 90% homologous or comprise only two or more preferably only one different amino acid residue, in a preferred embodiment the different amino acid residue is an amino acid, with similar charge or hydrophobicity with the amino acid in the sequence according to the invention.

### Analysis of Linkage Specificities

The invention is further directed to the testing of present antibodies and optional comparison antibodies with regard to binding to sialic acids, preferably NeuGc, comprising carbohydrates, which have different sialic acid linkage structures such as α3-, and/or α6-, and/or α8-linkage, and preferably controlling the experiment with corresponding NeuAc-comprising glycoconjugates. Preferred carbohydrates to be tested include any saccharides potentially comprising any of the terminal oligosaccharide sequences according to the invention and/or terminal oligosaccharide sequences not recognized by the antibodies. The invention is especially directed to the antibodies especially for the studies and analysis of all types of natural acid glycans, more preferably sialylated glycans and/or glucuronic acid comprising glycans and materials, even more preferably natural materials comprising these.

More preferably the carbohydrates to be analyzed are glycans on natural glycoconjugates such as on glycoproteins such as O-glycans and/or N-glycans or on glyco lipids such as glycosphingo lipids comprising glycans linked to ceramide. The terminal disaccharide or oligosaccharide epitopes recognized by the antibodies according to the invention are preferred as terminal oligosaccharide epitopes as part of glycans or when corresponding sequence is present as a whole (natural) glycan such as Tn antigen, the antibody recognizes preferably essentially whole glycan (at least partially all monosaccharide residues in the sequence) and optionally further part of the carrier structure. The oligosaccharide specificity allows analysis of the natural mammalian glycoconjugates, in a preferred embodiment the glyconjugates preferably glycoproteins and/or glyco lipid, are present on a cell and/or tissue material, more preferably on cell materials such as isolated cells and/cultivated cells.

### Preferred Production of Variants of the Present Antibodies

It is realized that similar human monoclonal antibodies can be produced by similar methods from human antibody phage display libraries. It is realized, that similar antibodies can be produced by changing single amino acid residues, which are not essential for the antibody binding, or can be changed to allow similar binding. The changing of amino acid residues is preferably performed by regular recombinant DNA technologies producing single mutations or producing libraries of mutated protein sequences and screening the sequences for the binding to NeuGc comprising carbohydrate structures. The invention is directed to the use of known similarity of certain amino acid residues such as residues with similar side chain properties such as hydrophilic/hydrophobic structure, size, charge, or aromatic structure, for design and/or production of the mutations and variants of the present antibodies.

The invention is directed to methods of defining the three dimensional structures of the antibodies by molecular modelling and/or X-ray crystallography and/or NMR-methods, preferably the structure is produced in complex with a specific saccharide or NeuGc-residue comprising carbohydrate structure. The invention is further directed to defining the complex and use the information for further designing experiments for mutagenesis of the antibody sequences and developing the specificity and/or affinity of the antibodies to specific saccharides and/or NeuGc comprising structures by mutagenesis of the amino acid residues of the antibody.

Due to human compatibility the natural sequences according to the invention and their possible close homologues from human antibody display libraries are preferred for various human uses e.g. in human in vivo or for in vitro diagnostics avoiding cross-reaction from human serum antibodies with alternative non-human antibodies. It also realized that the present antibodies or their ligand binding sequences in chimeric forms with animal antibody frame are preferred for use in animal trials in order to study the biological activities of the antibodies, having advance due to fact that the antibody sequences are recognizable from the natural antibodies of the test animal species.

For use in immunoassay, e.g. for qualitative or quantitative determination of saccharides and/or NeuGc in biological samples, antibodies and antibody derivatives of the invention may be labelled. For these purposes, any type of label conventionally employed for analytic or diagnostic antibody labelling is acceptable.

For use in immunotherapy, e.g. for targeting xenoantigenic NeuGc in malignant tissues in patients, antibodies and antibody derivatives of the invention may be labelled with a therapeutic molecule. For these purposes, any pharmaceutically acceptable label conventionally employed for therapeutic antibody labelling is appropriate.

For blocking of binding harmful NeuGc-recognizing antibodies, the antibody/antibody conjugate is preferably not cytotoxic. Non-immunogenic antibody fragments, such as Fab-fragments or scFv-type fragments, may be used for blocking binding of autoimmunity reaction suffering or transplanted tissue by natural NeuGc antibodies to antigenic NeuGc-structures.

For use in in vivo imaging, e.g., antibodies and antibody derivatives of the invention may be labelled. For these purposes, any pharmaceutically acceptable imaging label conventionally employed for antibody labelling is appropriate.

Numerous ways of conjugating antibodies and antibody fragments are known in the art. Typically antibody is conjugate at a site away from the antigen binding site. The conjugation is in a preferred embodiment performed from a glycan, preferably N-linked glycan of an antibody, such as a Fc-domain N-glycan or from a glycan produced to novel glycosylation site produced by mutagenesis. Other preferred sites of conjugation is N- or C-terminal of the polypeptide remote from the variable regions, preferably terminus comprise a structure which can be chemically modified, without harming the protein structure, such as N-terminal serine residue, which can be oxidized (similarly as glycans) and the conjugated specifically by aldehyde reactive reagents such as hydrazine or aminooxy-reagents, which are linked to therapeutic or diagnostic molecular structure. The therapeutic or diagnostic molecular structure is preferably a cytotoxic, or a radioactive, or a prodrug/prodrug releasing molecule for therapy; or for analytic uses e.g. an ELISA reagent, a photoactivable molecule for optical analysis, biotin for avidin/strepavidin labellings, or a radioactive or NMR/MRI-active molecule for in vivo imagining.

In another aspect, the present invention also provides DNA molecules encoding an antibody or antibody derivative of the invention, and fragments of such DNAs, which encode the CDRs of the V_{L} and/or V_{H} region. Such a DNA may be cloned in a vector, more particularly, for example, an expression vector which is capable of directing expression of antibody derivatives of the invention, or at least one antibody chain or a part of one antibody chain.

In a further aspect of the invention, host cells are provided, selected from bacterial cells, yeast cells, fungal cells, insect cells, plant cells and mammalian cells, containing a DNA molecule of the invention, including host cells capable of expressing an antibody or anti-body derivative of the invention. Thus, antibody derivatives of the invention may be prepared by culturing host cells of the invention expressing the required antibody chain(s), and either directly recovering the desired protein or, if necessary, initially recovering and combining individual chains.

The above-indicated scFv fragments were obtained by biopanning of a human IgM scFv-phage library using xenoantigenic recombinant NeuGc. The human IgM scFv-phage library was constructed from mRNAs isolated from lymphocytes of 50 healthy blood donors. The variable region of the light and heavy chain cDNAs were synthesised using human IgM-specific primers for Fd cDNAs and human kappa (κ) and lambda (λ) light chains using human κ and λ chain specific primers. The variable regions of the light and heavy chains were amplified by PCR using human κ and λ chain specific primers for Vκ and Vλ cDNAs and human IgM specific primers for V_{H} cDNAs, respectively. The human IgM scFv library was constructed by cloning the variable region cDNAs into a scFv phage display vector using restriction sites introduced into the PCR primers.

The human IgM scFv library was selected by phage display using a panning procedure. The human IgM scFv phage library was screened by a biotinylated xenoantigenic recombinant NeuGc in solution and the binders were captured on streptavidin. The elution of phages was done with 100 mM HCl (pH 2.2) followed by immediate neutralisation with 2 M Tris solution. The phage eluate was amplified in E. coli cells. After 4 rounds of biopanning, soluble scFv fragments were produced from isolated phages. The binding specificity of the selected scFv fragments was analysed by ELISA. Several saccharide and/or NeuGc-specific scFv fragment clones were obtained.

As described herein, the phage display technique is an efficient and feasible approach to develop human IgM recombinant anti-saccharide and/or anti-NeuGc antibodies for diagnostic and therapeutic applications.

While one successful selection strategy for obtaining antibody fragments of the invention has been described, numerous variations, by which antibody fragments of the invention may be obtained, will be apparent to those skilled in the art. It may prove possible to select scFv fragments of the invention directly from a phage or microbial display library of scFv fragment or its derivatives. A phage or microbial cell, which presents a scFv fragment or other antibody fragment of the invention as a fusion protein with a surface protein, represents a still further aspect of the invention.

While microbial expression of antibodies and antibody derivatives of the invention offers means for efficient and economical production of highly specific reagents of uniform quality suitable for use in immunodiagnostic assays and immunotherapy, alternatively it may prove possible to produce such a reagent, or at least a portion thereof, synthetically. By applying conventional genetic engineering techniques, initially obtained antibody fragments of the invention may be altered, e.g. new sequences linked, without substantially altering the binding characteristics. Such techniques may be employed to produce novel saccharide and NeuGc-binding hybrid proteins, which retain both affinity and specificity for saccharides and NeuGc as defined hereinbefore.

### Specific Methods for Selecting NeuGc-Antibodies

The invention is directed for the selecting of an antibody fragment from a phage display antibody library, when the display library of antibody fragments is selected as non-binding towards non-reducing end single terminal NeuAcα-conjugate and as the binding to non-reducing end single terminal NeuGcα-conjugate. Preferably the conjugates for the selection are immobilized. Even more preferably said NeuAcα-non-binding conjugates are first selected out of the phage library and then NeuGcα-binding clones are selected from the library. It is realized that antibody libraries can be constructed in various ways, in a preferred embodiment the library is a scFv-library.

The present invention revealed novel useful method of selecting an antibody fragment from a library of human antibodies, preferably form a library derived from multiple persons, more preferably the library is derived from at least about 50 persons. The library shown in the examples is derived from blood cells of about 50 healthy blood donors. Due to large number of donors the library is likely to contain practically all possible human antibodies against the single terminal NeuGcα-residues. The antibody libraries give same clones from multiple selections indicating that the method is reproducible.

The selection in the examples was performed from a library of IgM antibodies. The present invention is preferably directed to selection of IgM-antibodies for production/discovery of anti-NeuGc-antibodies. There are typically differences between IgM and other antibody types because of “maturation” antibodies. The IgM-antibodies are also naturally decavalent and the present selection method was designed to mimic the natural oligovalent recognition of NeuGc by using phages displaying the antibody fragments in oligovalent form. The invention indicates that the antibodies according to the present invention are useful for recognition of the polyvalent clustered saccharide or NeuGc-structures with polyvalent binders as naturel IgM but also for recognition of monovalent epitopes by FAb type reagents. The other antibody types are typically divalent and likely less useful for oligovalent recognition of antigens.

The antibody fragments are selected against polyvalent conjugates of NeuAcα and NeuGcα. For exact selection both structures are preferably conjugated to the same carrier structure. The invention is specifically directed to antibodies which can recognize clustered oligovalent epitopes of NeuGc. It is notable that previous works about NeuGc-recognizing antibodies are describe binding to unimolecular glycolipid structures, which contain single NeuGc-residue or two NeuGc-residues in structure very close to each other like in structure NeuGcα8NeuGcα3Galβ4GlcβCer. It is further realized that present antibodies are useful for recognition of the glycan structures also from other glycoconjugates than glycolipids as the screening was performed against the non-reducing end terminal structure.

The preferred polyvalent conjugates have a distance between sialic acid residues of less than about 20 atomic bonds but more than about six atomic bonds. The polyvalent polyvalent conjugate comprises preferably flexible polyamide structure, more preferably a polyacrylamide structure. Flexible indicates that the structure comprises spacers with methylene structures. Preferably NeuAcα/NeuGcα is linked to three carbon spacer, being preferably a methylene-radical, further conjugated to the polyacrylamide back bone. Polyvalent acrylamide conjugates can be synthesized chemically as described by Bovin N. 1998, polyvalent polyacrylamide conjugates are commercially available from reagent supplier such as Sigma Co. St Louis, USA or Syntesome, Russia.

The invention further directed to antibodies discovered by selection from human antibody libraries according to the invention.

Novel Specificity Characteristics of the Saccharide and/or NeuGc Antibodies According to the Invention

The antibodies according to the invention revealed binding specificity to xenoantigenic non-reducing end single terminal NeuGcα, but not binding non-reducing end single terminal NeuAcα, when analyzed with polyvalent monosaccharide conjugates. It is notable that certain antibodies recognize sialic acids in non-terminal positions such as in oligo- or polysialic acids NeuGcα8NeuGcα3Galβ4GlcβCer, and not as terminal non-reducing end residues, for example the antigens used for purification of chicken polyclonal antibodies of Varki and colleagues were truncated with regard to the glycerol structure and thus would allow recognition of terminally modified and/or elongated NeuGc-structures. The present invention revealed good binding active antibodies selected for non-reducing terminal NeuGcα. The effective recognition does not require additional modifications but it is affected by the carrier structures or elongation by other NeuGc-residue, and thus the epitope is referred as single terminal NeuGcα, including the terminal monosaccharide residue conjugated to a carrier.

The antibodies have binding specificity, which allows recognition of human cells containing certain acidic saccharides and/or non-reducing terminal NeuGc epitopes. This is in contrast to previously published human antibodies one of which did recognize terminal non-reducing end NeuGc on glycolipids but not on human cells and one which did not recognize terminal non-reducing end NeuGc on glycolipids but apparently not on proteins but bound human cells grown in fetal bovine serum.

Furthermore the invention describes for the first time phage display or other human antibodies capable of effectively recognizing a single terminal monosaccharide with only minor variation of one proton substituted by a hydroxyl group such as in antibodies binding to NeuGc-terminal monosaccharide residue but not to NeuAc-terminal monosaccharide residue. The high monosaccharide level selectivity has not been described for human or any other NeuGc antibody selected for binding complete NeuGc-residue. The unique monosaccharide selectivity was further studied with two other human terminal monosaccharide residues conjugated as the sialic acids and with difference of single epimeric position, which did not yield similar selective antibodies.

It is realized that the phage display system produces natural type human antibodies. These should be more easily acceptable for human use than animal antibodies or humanized animal antibodies, which contain structures unnatural in human.

Furthermore the antibodies of the present invention recognize effectively polyvalent high density/clustered conjugate of NeuGc (described above) and it is in a preferred embodiment used in a clustered oligovalent form such as in tri- to decavalent forms mimicking the recognition of human IgM or produced as human IgM-antibody by methods known in the art, e.g. Volmers et al., OncoMAb™, Germany. Surprisingly the antibodies are also effective as monovalent Fab type or single chain antibodies, though in general the affinities of the FAbs IgM antibodies recognizing glycans are very low. It is notable that most of the antibodies in background are of different type involving different specificity and usually only divalent structures.

### Analysis of Nucleic Acids

The invention is further directed to nucleic acid sequences corresponding to the antibody sequences including all variants of genetic code. These are well-known to any person skilled in the art. The present invention is especially directed to the human natural nucleic acid sequences coding the antibodies. The invention is further directed to the complementary nucleic acid sequences for the human natural nucleic acid sequences. The invention is further directed to the use of the nucleic acid sequences and the complementary nucleic acid sequences and homologues thereof with the similar capacity to bind and hybridize with the nucleic acid sequences a) for analysis of expression of the nucleic acid sequences b) for effecting the expression of the nucleic acid sequences. A preferred group of preferred nucleic acid homologues includes peptide nucleic acids. The preferred nucleic acid sequence analysis includes cloning and sequencing of the nucleic acid sequences, and analysis by hybridization methods and by PCR-methods such as RT-PCR methods.

The invention is especially directed to the analysis of the nucleic acid in context of analysing a human immune reaction against specific saccharides and/or NeuGc, preferably in the context of immune reaction against transplant, more preferably in context of cell transplant or xenotransplant, when there is reason to believe that the transplanted material comprise specific saccharides and/or NeuGc. The invention is further directed to the analysis of the nucleic acids according to the invention from a person in the context of a nutritional change in the amount of NeuGc in food.

### Analysis of Cells and Tissues

The development and characterisation of the specific saccharide and/or human NeuGc-binding recombinant antibodies and their usefulness in immunoassays is now described in more detail in the following examples. The invention is specifically directed to the use of the antibodies for analysis of cells and tissues. Preferred cells and tissues to be analyzed include cell materials of animal origin or materials, which have been in contact with animal material containing NeuGc. The invention revealed that the present antibodies are useful and preferred for analysis of acidic glycans and/or NeuGc-structures of the invention from animal cell or animal cell/tissue derived materials such as pig cells or proteins.

### Preferred Antibody Specificities, Sequences and Methods

The invention is especially directed to human monoclonal antibody that binds to terminal non-reducing end oligosaccharide sequences:
1) α3-sialylated type 1 N-acetyllactosamine sequence SAα3Galβ3GlcNAc, wherein SA is Neu5Gc or Neu5Ac, said sequence being preferably Neu5Gcα3Galβ3GlcNAc. It is realized that the recognition of type 1 N-acetyllactos amine with Neu5Gc is very unusual and useful property for an antibody, especially incontext of recognition of materials, which may contain the xenoantigenic (non-human) sialic acid Neu5Gc).
and/or
2) α6-sialylated type 2 N-acetyllactosamine sequence SAα6Galβ4GlcNAc, wherein SA is Neu5Gc or Neu5Ac. The invention especially revealed binding to the epitope wherein the sialyl-lactosamine is not linked to a N-glycan structure and strong or practically exclusive recognition, when the sialic acid is Neu5Gc. These are quite unusual characteristics for an antibody oligosaccharide binding, but the specificity is most preferred with the other specificities.
and/or
3) sialylated non-reducing end terminal Neu5Acα6GalNAc-structures, preferably sialyl-Tn sequences Neu5Acα6GalNAcα.
and/or
   terminal non-reducing end monosaccharide residues:
4) xenoantigenic non-reducing end single terminal NeuGcα-monosaccharide residue, but does not bind to non-reducing end single terminal NeuAcα-monosaccharide residue,
   and preferably does not bind to
5) oligosaccharide sequences according to SAα3Galβ4Glc(NAc)ₙ, wherein SA is Neu5Gc or Neu5Ac and n is 0 or 1.

It is realized that none of the oligosaccharide sequences has been characterized as cell culture condition dependent markers of human stem cells or specific subtypes thereof.

The invention is especially directed to the unique antibodies with specificities combining the preferred oligosaccharide binding specificities. In a preferred embodiment the specificites includes at least
terminal non-reducing end oligosaccharide sequences:
1) α3-sialylated type 1 N-acetyllactosamine sequence SAα3Galβ3GlcNAc, wherein SA is Neu5Gc or Neu5Ac, said sequence being preferably Neu5Gcα3Galβ3GlcNAc and
2) SAα6Gal(NAc)n, wherein SA is sialic acid, preferably being Neu5Gc or Neu5Ac and n is 0 or 1. The second group represent similar a6-linked sialic acid structures, which is unusual specificity together with the α3-sialic acid binding.

The preferred binding to oligosaccharide sequences SAα6Gal(NAc)n includes α6-sialylated type 2 N-acetyllactosamine sequence SAα6Galβ4GlcNAc, wherein SA is Neu5Gc or Neu5Ac, and

sialylated non-reducing end terminal Neu5Acα6GalNAc-structures, preferably sialyl-Tn sequence Neu5Acα6GalNAcα.

The specificity is further characterized by specificity with regard to polymer conjugated sialic acid residues and non-binding or very low binding activity oligosaccharide sequences as shown in examples, especially including a3-sialylated lactose and type II N-acetyllactosamine. Terminal non-reducing end monosaccharide residues further include:
1) xenoantigenic non-reducing end single terminal NeuGcα-monosaccharide residue, but said antibody does not bind to non-reducing end single terminal NeuAcα-monosaccharide residue linked from reducing end to a polymer carrier,
   and the antibodies preferably do not bind to common sialyl-lactosamine oligosaccharide sequences
2) oligosaccharide sequences according to SAα3Galβ4Glc(NAc)ₙ, wherein SA is Neu5Gc or Neu5Ac and n is 0 or 1.

In a preferred embodiment several major specificity characteristics are included and the preferred antibody binds to both α3-sialylated type 1 N-acetyllactosamine sequences Neu5Gcα3α3Galβ3GlcNAc, and Neu5Acα3α3Galβ3GlcNAc, and

wherein the antibody binds to terminal non-reducing end epitopes sialyl-Tn sequences Neu5Acα6GalNAcα, and
wherein the antibody binds to both α6-sialylated type 2 N-acetyllactosamine including Neu5Acα6Galβ4GlcNAc, and Neu5Gcα6Galβ4GlcNAc,
and wherein the antibody binds to terminal non-reducing end epitopes Neu5Acα6Galβ4GlcNAc with higher affinity than Neu5Gcα6Galβ4GlcNAc, and/or more effectively to Neu5Gcα3Galβ3GlcNAc than Neu5Acα3α3Galβ3GlcNAc and/or not to Neu5Gcα6GalNAcα.

The invention is further directed to a monoclonal antibody, wherein the antibody binds to α3-sialylated type 1 N-acetyllactosamine sequence SAα3Galβ3GlcNAc, wherein SA is Neu5Gc or Neu5Ac, preferably more effectively Neu5Gcα3Galβ3GlcNAc; and/or wherein the antibody binds to both Neu5Gcα3Galβ3GlcNAc and Neu5Acα3Galβ3GlcNAc

The invention is further directed to a monoclonal antibody, wherein the antibody binds to α6-sialylated terminal non-reducing end epitopes according to the formula SAα6Gal(NAc)n, wherein SA is sialic acid, preferably being Neu5Gc or Neu5Ac

The invention is further directed to a monoclonal antibody, wherein the antibody binds to terminal non-reducing end epitopes Neu5Acα6GalNAc, preferably sialyl-Tn sequences Neu5Acα6GalNAcα.

The invention is further directed to a monoclonal antibody, wherein the antibody binds to both α6-sialylated type 2 N-acetyllactosamine including Neu5Acα6Galβ4GlcNAc, and Neu5Gcα6Galβ4GlcNAc.

The invention is further directed to a monoclonal antibody, wherein the antibody binds to terminal non-reducing end epitopes Neu5Acα6Galβ4GlcNAc with higher affinity than Neu5Gcα6Galβ4GlcNAc. The affinities are in a preferred embodiment measured by ELISA assay as described in the invention.

The invention is further directed to a monoclonal antibody, wherein the antibody binds to terminal xenoantigenic non-reducing end single terminal NeuGcα-monosaccharide residue, but does not bind to non-reducing end single terminal NeuAcα-monosaccharide residue

The invention is further directed to a monoclonal antibody, wherein the antibody does not bind to oligosaccharide sequences according to SAα3Galβ4Glc(NAc)ₙ, wherein SA is Neu5Gc or Neu5Ac and n is 0 or 1.

### Preferred Polypeptide Sequences

The invention is directed to the antibody, which has preferred polypeptide sequences according to the invention. The antibodies further preferably have the binding specificity characteristic(s) according to the invention. The invention revealed novel useful antibodies for recognition of oligosaccharide sequences. The antibodies have special usefulness for therapeutics and diagnostics because they are human antibodies and are not effectively recognized by human immune system.

### Antibody CDR Sequences

It is realized that the CDR sequences are a characteristic for the antibody family and simila antibodies can be recognized base on fragments of full CDR sequences of the antibodies. The invention is further directed to at least 40%, more preferably at least 50%, even more preferably at least 60%, more preferably at least 70%, more preferably at least 80%, and even more preferably at least 90%, similar or more preferably identical antibody sequences. The similar sequences are especially preferred for methods of searching new antibodies with same or similar specificites as the antibodies according to the invention or for optimization of an antibody according to the invention, e.g. by mutagenesis methods and screening the resulting antibodies against the preferred oligosaccharide sequences according to the invention.

The invention is further directed to short characteristic epitopes include tri- to decapeptide fragments of the preferred consensus sequences.

### Preferred Short Sequences

The preferred heavy chain sequences of the antibody polypeptides comprise heavy chain sequences of 1.4. group antibodies with CDR1 sequences consensus sequence CDR1: GFTFR, GFTFS, GITFR, or GITFS; FTFR, FTFS, ITFR, or ITFS;

or

CDR1: X₁TFX₂X₃Y

wherein X₁ is preferably I or F; and X₂ is R or S; and X₃ is K, or S, or R;
and/or with
CDR2 sequences having preferred short consensus sequence is YADSVK or YYAD, YYADS, YYADSV, YADS, or YADSV. The sequences with two tyrosines are especially preferred as characteristic peptides.

Further preferred CDR1 fragments include TFRK, TFRKY, TFRKYA, TFRKYAM, TFRKYAMN, TFSS, TFSSY, TFSSYA, TFSSYAM, TFSSYAMS, TFSR, TFSRY, TFSRYS, TFSRYSM, TFSRYSMN; FRKY, FRKYA, FRKYAM, FRKYAMN, FSSY, FSSYA, FSSYAM, FSSYAMS, FSRY, FSRYS, FSRYSM, FSRYSMN; RKYA, RKYAM, RKYAMN, SSYA, SSYAM, SSYAMS, SRYS, SRYSM, and SRYSMN. The shorter fragment or epitopes are especially preferred for methods of searching or optimizing new antibodies, For these methods tripeptides are most preferred the tetra-, penta, and hexapeptides and larger ones in order of decreasing preference. Preferred tripeptides includes TFS, and TFR; FRK, FSS and FSR; RKY, SSY, and SRY.

The invention is further directed to an antibody, which has the binding specificity characteristics according to the invention and which comprises heavy chain CDR1 and CDR2 sequences of 1.4. group antibodies with consensus sequence:

### CDR1: X₁TFX₂X₃YX₄MX₅,

wherein X₁ is preferably I or F; and X₂ is R or S; and X₃ is K, or S, or R; and X₄ is A or S; and X₅ is N or S.

### CDR2: X₁₁SX₂SX₃X₄X₅X₆YYADSVKG,

wherein X₁ is preferably A or S; and X₂ is N, G, or S; and X₃ is G, or S; and X₄ is S or G; and X₅ is D, S or Y; and X₆ is T or I,
and optionally
CDR3: X₁X₂X₃X₄X₅X₆X₇DX₈, wherein X₁ is preferably R or M: and X₂ is P, K or N and X₃ is K or nothing; and X₄ is G or nothing; and X₅ is G, A, or nothing; and X₆ is G, or A; and X₇ is M, or F, and X₈ is V, or P or I,
or
heavy chain CDRs of 1.2.20 type antibodies:

CDR1: GTVNSYYWS,
CDR2: RVYSSGTTNLNPS,
CDR3: DYGTDY

The invention is further directed to antibodies, wherein the antibody comprises light chain CDR1 and CDR2 sequences of 1.4. group antibodies with consensus sequences:

CDR1: TLRSG or TLRSGINVGX₁X₂RIY, wherein X₁ is preferably A or T and X₂ is Y or S
CDR2: KSX₁SDKQQGS, wherein X₁ is preferably N or D, and
optionally
CDR3: MIWHX₁X₂AX₃WV, wherein X₁ is preferably S or N and X₂ is G or R and X₃ is W or V
or
1.2.20 type antibody with sequence:

CDR1: GGDNL, GGDN, GDNL, or GGDNLGGKSVH,
CDR2: DDRDRPS,
CDR3: QVWDSGSESVV.

The preferred short characteristic epitopes include tri to- decapeptide fragments of the preferred consensus sequences, preferably for light chain CDR1 including: TLRS, TLRSG, TLRSGI, TLRSGIN, TLRSGINV, TLRSGINVG, LRS, LRSG, LRSGI, LRSGIN, LRSGINV, LRSGINVG, RSG, RSGI, RSGIN, RSGINV, RSGINVG, SGI, SGIN, SGINV, SGINVG, GIN, GINV, GINVG, INV, INVG, and NVG.

A preferred antibody comprises at least one of the 1.4 type light chain CDR sequences, preferably at least two being preferably CDR1 and CDR2 and most preferably all sequences CDR1-3.

It is further realized that novel antibodies can be produced by combining the light chain and heavy cahin sequences, or homologous sequences of the antibodies according of the invention, in a preferred embodiment the antibody comprises the light chain CDR1-CDR3 sequences selected from the group 1.4.11, 1.4.24 sequences 1.4.30, or 1.2.20:

and heavy chain CDR1-CDR3 sequences selected from the group 1.4.11, 1.4.24 sequences 1.4.30, or 1.2.20. More preferably sequences of 1.4-group antibodies are combined, e.g. as in 1.4.19-3 (F3) antibody. It is realized that any of CDR1, CDR2 or CDR3 can be derived from different original sequences.

A preferred antibody comprises the light and heavy chain CDR1-CDR3 sequences of 1.4.24 antibody and in a preferred embodiment 1.4-group light chain sequences, in a preferred embodiment the 1.4.24 light chain sequences. The 1.4.24 and 1.4.19 (−3), more preferably 1.4.24 antibodies are preferred for their higher affinities to oligosaccharide sequences. This was shown in examples by ELISA assay, the invention is especially directed to the antibody specificties, wherein the specificities are compared by elisa assay using polyvalent oligosaccharide conjugates, preferably polyacrylamide conjugates.

### Analysis Methods

The invention is directed to a method of analysis of disease associated or a cell binding antibody, preferably human antibody, wherein the method includes step of measuring the specificity of the antibody towards the sialylated oligosaccharide and monosaccharide sequences according to the invention, preferably using oligosaccharide sequences shown in examples, preferably measuring specificity with regard 3 oligosaccharide sequences of included in the preferred binding specificity, preferably the preferred a3- and a6-linked sialyl-oligosaccharide sequences. Preferably the specificity is measured when antibody has sequence or sequence fragment according to the invention or homologous sequence or at least one similar or homologous CDR1-3 sequence.

The invention is further directed to methods for searching or characterizing or optimization of antibodies including a method for detecting carbohydrate epitope binding antibodies, the method comprising the steps of:

a) searching from available sequence data antibody sequences having essentially similar or same CDR1 or CDR2 sequences or sequence fragment or homolog as described in the invention;
b) contacting an antibody found in step a) with sialyl saccharide library comprising saccharide sequences as described in the preferred saccharide binding specificity according to the invention;
c) detecting if said antibody binds to any of said saccharide sequences or in preferred embodiment have the same binding specificity as the antibody according to the invention.

The invention is especially directed to the selection of antibodies having essentially same or qualitatively similar specificity including binding to the same oligosaccharide sequences, preferably in the ELISA assay according to the invention.

The sequence data may be available from sequence databases or from sequencing of antibodies as known in the art.

Analysis of Cultivated Cells or Cells which have been in Contact with Exogenous Materials

### Preferred Cells Types and Analysis Methods

The invention is especially directed to a method to analyze status of human cells, to analyze status of a human stem cell population involving a step of contacting the cells with a binder reagent, preferably a monoclonal antibody, according to the invention, for the analysis of a effect of exogenous materials and/cell culture conditions to the cells.

The analysis method is especially directed to the cell surface expression of glycan structures on an intact cell population. It is realized that it is useful to analyze cell surface structures, which are most relevant with regard to immunological responses in vivo and or cell biology of the cells, preferably stem cells.

The labelling of the human cells, preferably human stem cells, by the antibody is associated with cell culture conditions in the presence of non-human exogenous material and/or lack of the labelling is associated cell culture conditions in the presence of human equivalent material. It is realized that non-human materials, also referred as exogenous or xenoantigenic materials, can be used in cell cultures and changes or contaminations by these to the cells would affect the suitability of the cells for human in vivo uses, for example, by alterations immunological suitability and/or cell biological targeting properties of the cells.

The non-human exogenous materials preferably comprise non-human or animal type glycan structures in said non-human exogenous materials, preferred non-human exogenous materials are non-human animal proteins/peptides used in cell culture such as animal serum preoteins or animal cellular proteins, preferably animal serum proteins such as animal serums or fractons thereof such as FCS (fetal calf serum) or animal cell preparations (e.g. pig cell preparations) or recombinantly produced proteins derived from cell culture producing non-human glycan structures. The human equivalent materials, which are associated with the lack of labelling mean in a preferred embodiment the presence of human type glycan structures in said human equivalent materials (and preferably non-presence of animal type glycans), such as human serum or cell/blood cell derived proteins such as human serum proteins and/or recombinant human proteins produced to comprise human glycosylation.

The invention is directed to the analysis methods, wherein major subpopulation of the intact cells is labelled, more preferably at least 15%, even more preferably at least 20%, even more preferably at least 25%, %, even more preferably at least 35%, even more preferably at least 45%, even more preferably at least 55%, even more preferably at least 65%, and most preferably at least 75% or 80 m % of the cells are labelled.

The invention reveled that unexpectedly large portion of the human cells, preferably human stem cells, most preferably human mesenchymal stem cells according to the invention are labelled by the novel reagents. The preferred stem cells are human blood derived mesenchymal stem cells, more preferably cord blood or bone marrow derived mesenchymal stem cells.

The invention is especially directed to the analysis method according to the invention, wherein novel antibodies according to the invention are used.

Most preferred cells to be analyzed include
i) cultivated cells,
and/or
ii) cells, which have been in contact with exogenous carbohydrate materials such as serum and/or exogenous glycoproteins and/or glycolipids
and/or
iii) cells which have grown in conditions inducing the expression of one or more of the specific oligosaccharide recognized by the antibodies according to the invention.

The inventors have been previously involved in revealing alteration of cell glycosylation based on, even very brief, exposure of exogenous carbohydrate materials such as animal derived low purity albumin preparations, or cell sorting reagents such as Fc blocking reagent in magnetic sorting system. It is further known that cell culture condition can induce expression of novel glycans e.g. by providing precursor materials (e.g. sialic acids such as Neu5Gc or glyco lipids) for biosynthesis of special oligosaccharide sequences on cell surfaces and/or by affecting the control of glycan biosynthesis in cells.

### Intact Cells

The present invention revealed that the antibodies can recognize saccharide sequences on intact cells observable by flow cytometry such as FACS analysis and/or immunohistochemistry. The present invention is especially directed to analysis of one or more the saccharide sequences, more preferably oligosaccharide sequences on intact cells, more specifically as antibody accessible material.

### Cell Culture

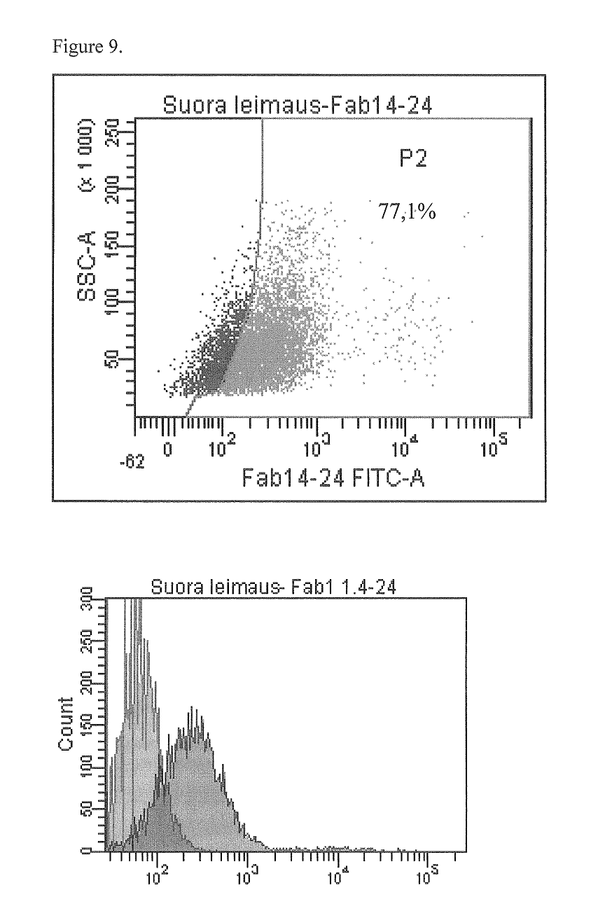

FIG. 9 shows labelling of human cord blood mesenchymal stem cells, human CB-MSC cells, by 1.4.24 antibody in FACS (fluorescence activated cell sorting). The cells were cultivated in presence of exogenous non-human materials, and the labelling was not observed when the non-human material were replaced by “xeno-free materials” or more specifically human derived materials. The data shows a major population of intact cells labelled by the antibody, and the labelling does depend on cell culture conditions. Neuraminidase (sialidase) treatment was used to confirm the sialic acid dependent binding to the cells. The example further shows effective labelling of human stem cells when being in contact with exogenous (non-human) materials and effective labelling of animal cells.

In a preferred embodiment the invention is directed analysis of cultivated cells with regard to contamination by exogenous materials, more preferably material comprising or inducing presence of one or more of the oligosaccharide sequences recognized by the present antibodies.

In a preferred embodiment the antibodies are used to analysis of cells cultivated in presence of non-human animal materials such pig or cow derived material, preferably when the material comprises one or more of the oligosaccharide sequences according to the invention.

Most preferred cells to be analyzed include cultivated cells, preferred cell types include cells known to incorporate NeuGc(Neu5Gc), especially when these have been in any contact with NeuGc-containing biological materials. It is further known that not all cells are effectively contaminated by NeuGc. The inventors have in copending applications revealed that specific sialylated glycan structures can be incorporated to hematopoietic, mesenchymal or embryonic stem cells. The invention is in a preferred embodiment especially directed to evalution of cells comprising NeuGc in context of specific oligosaccharide sequences recognized by the present antibodies.

Preferred cell types to be analyzed include human cells, more preferably human stem cells, even more preferably human hematopoietic cells, bone marrow derived cells, cord blood cells, mesenchymal stem cells and embryonal stem cells or other stem cells and like and possible feeder cells for these cell types, especially when these have been in any contact with NeuGc-containing and preferred sialyl-oligosaccharide sequence containing or materials inducing presence of specific oligosaccharide sequences.

It is realized that the present antibodies can be used for recognizing various contamination or contamination induced oligosaccharide sequences on the preferred cell types.

The inventors have specifically found novel possibilities for effective NeuGc contaminations and/or Neu5Gc/sialic acid comprising oligosaccharide contamination, from multipotent cells, preferably these are multipotent cells, which are not of embryonal origin, more preferably the cell types include hematopoietic cells, bone marrow derived cells, cord blood cells, and mesenchymal stem cells, which are all of good therapeutic potential and with less teratocarcinogenesis type risks as have the embryonal stem cells. It is further known that not all cells are effectively contaminated by NeuGc or sialic acid oligosaccharide comprising glycoconjugates.

### REFERENCES

Bovin, N. V. (1998) Glycoconjugate J. 15, 431-446
Furukawa, K., Yamguchi, H., Oettgen H. F., Old L. J., and Lloyd K. O. (1988) J. Biol. Chem. 263, 18507-12.
Heiskanen, A., Tero Satomaa, T., Tiitinen, S., Laitinen, A., Mannelin, S., Mikkola, M., Olsson, C., Miller-Podraza, H., Blomqvist, M., Olonen, A., Lehenkari, P., Tuuri, T., Otonkoski, T., Natunen, J., Saarinen, J. & Laine, J. (2006) submitted.
Kabat, E. A., Wu, T. T., Reid-Miller, M., Perry, H. M., and Gottesman, K. S. (1991) Sequences of proteins and immunological interest, 4^{th} Ed., U.S. Dept. of Health and Human Services, Bethesda, Md.
Moreno E. et al., Glycobiology (1998) 8 (7) 695-705

### EXAMPLES

### The recombinant NeuGc-Specific scFv Fragment by Phage Display Selection

In this example the human IgM scFv library was constructed and selected by xenoantigenic NeuGc in order to isolate scFv fragments with affinity and specificity to NeuGc monosaccharide. Construction of human IgM scFv phage library was prepared indirectly by constructing IgM Fab-κ and Fab-λ libraries first, and then the particular library DNAs were used for PCR amplification of variable domains of heavy and light chains.

Construction of naïve human IgM scFv libraries. Heparinised blood samples (10 ml) from 50 healthy blood donors were pooled and lymphocytes were isolated using the Ficoll-Plaque (Pharmacia) isolation protocol according to manufacturer's instructions. Total RNA was isolated from the human lymphocyte pool originating using Promega's RNAgents Total RNA Isolation kit according to the manufacturer's protocol. The first strand cDNA synthesis was carried out using Promega's Reverse Transcription system kit. The cDNAs encoding human IgM VH and VL regions were amplified with the VentPol (Biolabs) using the PCR-primers of Table 3. The final PCR products of the antibody fragments were pooled and digested with appropriate restriction enzymes. Digested DNA fragments, encoding VH region and Vκ and Vλ regions, were ligated into a phagemid vector and transformed into E. coli XL-1 Blue cells to yield scFv-κ and scFv-λ libraries of about 10⁸ independent clones.

Both the biotinylated panning (Ag+) and depletion (Ag−) antigens were coupled onto the streptavidin-conjugated magnetic beads (Dynal) according the manufacturer's protocol. The Ag+ was polyvalent Neu5Gcα-polyacrylamide-biotin and Ag− was polyvalent Neu5Aco-polyacrylamide-biotin both from Syntesome/Lectinity, Russia. The conjugate has 3-carbon alkyl spacer which is linked to branched polyacrylamide conjugate containing biotin branches.

Selection of the human scFv libraries. The human scFv-κ and scFv-λ libraries were selected by the phage display technique (McCafferty et al, 1990, Barbas et al, 1991). For isolation of NeuGc-specific fragments, the human naïve IgM scFv-κ and scFv-λ libraries were displayed on the surface of the bacteriophage in a multivalent format, the libraries were pooled and panned using an affinity panning procedure. Biotinylated polyacrylamide-conjugated sialic acid derivatives were coupled to streptavidin-conjugated magnetic beads (Dynal) according the manufacturer's protocol. A NeuAc conjugate (Ag−) was used for depletion and a NeuGc conjugate (Ag+) for panning of the library.

First the phage pools were allowed to react with the magnetic beads coupled with the depletion Ag (Ag−) that was used also as a background control in screening steps for 16 h at +4° C. Thereafter, the phage pools were withdrawn and transferred onto the beads containing either panning antigen (Ag+) or depletion antigen (Ag−, background). After a 2-h incubation at room temperature (RT), the beads were washed 2 times with PBS (10 mM sodium phosphate, pH 7.2, 140 mM NaCl) containing 0.05% Tween 20 and the bound phages were eluted with acidic buffer (100 mM Glycine-HCl, pH 2.2), and immediately neutralised with 2 M Tris solution. For the next panning round the eluted phage pools were amplified by infecting E. coli XL-1 Blue cells. For the multivalent display of the antibody fragments on a phage the hyperphage (Progen) was used in all panning rounds. Four rounds of panning were performed.

Soluble monovalent scFv-pIII fusions from the second, third and fourth panning round were expressed in E. coli XL-1 Blue cells. 148 individual clones were grown in a 1-ml scale for preliminary characterisation. The supernatants were analysed on ELISA using Ag+-coated wells to catch the glycan-specific binders and Ag−-coated wells to see the non-specific binding. Twelve most promising clones were sequenced and as a result six different DNA sequences were found. Five of them were selected for further characterisation in cell binding assays.

Characterisation of the specific saccharides and/or NeuGc-binding antibodies. Cell binding of the five monoclonal multivalent phages was studied by immunofluorescence staining of NeuGc-positive pig kidney tubular cells (LLC-PK1). The cells were grown on coated glass 8-chamber slides (Lab-TekII, Nalge Nunc, Denmark) in M199 cell culture medium supplemented with 5% fetal bovine serum (FBS), 100 units/ml penicillin and 100 μg/ml streptomycin at 37° C. under humidified atmosphere of 95% air and 5% CO₂ for 2 to 4 days. The cells were rinsed 5 times with PBS and fixed with 4% paraformaldehyde in PBS for 10-15 min at RT, followed by washings 3 times for 5 min with PBS. The non-specific binding sites were blocked with 3% HSA (human serum albumin, FRC Blood Service, Finland) in PBS for 30 minutes at RT.

Phage antibodies were diluted to 10⁶ pfu/ml in 1% HSA-PBS and incubated for 60 minutes at RT, followed by washings 3 times 10 min with PBS. Secondary murine anti-phage antibody (αM13, 1:500, Amersham) and tertiary FITC-labelled goat-anti-mouse (1:300, Sigma) antibodies were incubated for 60 min at RT, washed 3 times 5-10 min with PBS and mounted in Vectashield mounting medium containing DAPI stain (Vector Laboratories, UK). A non-specific hyperphage was used as a negative control. Specificity of the binding was tested by removing sialic acids from the cell surface by sialidase treatment before incubation with the phage antibodies. Four clones were identified which specifically bound to the cell surface but loosed their binding activity after sialidase treatments of the cells (Table 1).

Cloning of the human Fab fragments with glycan-binding specificity. The four human IgM scFv clones were selected for the conversion to human Fab fragments with IgG1 subtype (Holliger et al., 1993, Desplancq et al., 1994). The Fd regions and light chains were amplified by overlapping PCR using the primers of Table 4. The resulting cDNAs of the Fd region and light chains were cloned into the bacterial expression vector, pKKtac and then transformed into E. coli RV308. Soluble Fab fragments designated as 1.2.20, 1.4.11, 1.4.24 and 1.4.30 were produced.

The antibody Fab fragments were tested in immunostaining of sialylated cells and NeuGc comprising cells. Positive staining depending on sialic acids, releasable by sialidase enzyme, were observed when the antibodies were characterized with animal cellular materials, see Table 1. The cells were observed with Zeiss Axioskop 2 plus fluorescence microscope (Carl Zeiss Vision GmbH, Germany) with fluorescein and DAPI filters. Images were taken with Zeiss AxioCam MRc camera and with AxioVision Software 3.1/4.0 (Carl Zeiss) with 400× magnification. Intensity of the stainings was graded as—(negative) or +/++/+++ (positive).

The antibodies were also tested in Western blot assays. The assays indicated binding to glycoproteins.

### Comparison of Antibody Sequences

The antibody sequences according to the invention are compared with other available antibody sequences by standard methods. For example homologous sequences are searched by BLAST-program, which is available for example from entrez-netpages. Table 5 shows random examples of sequences which can be found by searching short nearly homologous sequences by BLAST with the specified sequences.

Part of the sequences are homologous or even identical with numerous antibody sequences, while part of the sequences, especially CDR3-sequences appear to be quite unique. The invention revealed rare or unique single amino acid residue mutations such as
1) the X₁-amino acid residue in light chain CDR2 of 1.4.30, N-residue was not found in any other antibody
2) X₂-amino acid residue in light chain CDR3: of 1.4.30, and 1.4.11: the G-residue was not found in any other antibody; and of 1.4.24, where it is R next also to rare X1-residue N
3) Rare L-residue on heavy chain CDR2 of 1.2.20 RVYSSGTTNLNPSLKS.

The CDR3 sequences have other rare characteristics. The heavy chain CDR3s are relatively short: 1.2.20 and 1.4.24 have 6 and 1.4.30 7 amino acid residues, and even 1.4.11. with 9 residues is relatively short. The heavy chain CDR3s appear also to have rare sequences, e.g. 1.2.20 heavy chain CDR3 was not found in any immunoglobulin. The invention is directed to the unique characteristic features and combination thereof with the more conserved corresponding CDR1 and 2-sequences and consensus sequences.

### Specificities of the Antibodies 1.4.24 and 1.4.30

### Experimental Procedures

Specificity of the antibodies 1.4.24 and 1.4.30 determined by immunoassay. Polyacrylamide(PAA)-biotin-conjugated polyvalent monosaccharides or glycans (Lectinity, Russia, see Table 6) were immobilized onto streptavidin microtiter plates (Perkin Elmer, Finland) 100 ng/well in TBS buffer (20 mM Tris-HCl, pH 7.5, 150 mM NaCl) at +4° C. o/n. Wells were washed 4 times with TBS and non-specific binding sites were blocked with 1% ultra pure BSA-TBS (Sigma, A7638) for 60 minutes at room temperature (RT). Antibodies 1.4.24 and 1.4.30 were diluted 3 μg/ml in 0.1% ultra pure BSA-TBS and incubated for 2 hours at RT. Furthermore, wells were washed 4 times with TBS and secondary antibody, Europium-labelled goat anti-human lambda (Southern Biotechnology) was diluted 1 μg/ml in 0.1% ultra pure BSA-TBS and incubated for 60 minutes at RT in the dark. Wells were washed as previously and 200 μl of DELFIA Enhancement solution (Perkin Elmer, Finland) was added per well, after which the plate was shaked for 5 minutes at RT. Europium signals were detected with Victor plate reader (Perkin Elmer, Finland).

### RESULTS AND DISCUSSION

Specificity of the antibodies. Both antibodies 1.4.24 and 1.4.30 are highly specific for NeuGc-monosaccharide (GF309) over naturally occurring NeuAc-monosaccharide (GF308, FIG. 8). However, both antibodies cross react with some acidic monosaccharides, such as glucuronic acid α/β (GF341 and GF271, respectively). Furthermore, anti-NeuGc antibodies show variable recognition of di- and tri-monosaccharides carrying either NeuGc or NeuAc monosaccharide. When sialic acid (SA) is linked with α2-6 linkage to either N-acetyl galactosamine (GalNAc) or galactose (Gal), NeuAc is recognized at least 4 times better than NeuGc (GF345-GF348). When SA is α2-3 linked to type 1 LacNAc, both NeuGc/Ac are recognized by antibodies, NeuGc slightly better than NeuAc (GF462 and GF461, respectively). All other structures, where SAs are linked with α2-3 linkage (GF459, GF460, GF463-GF468) are not recognized at all by anti-NeuGc antibodies.

Testing Fab Fragment with Human Stem Cells

The antibody Fab fragments were tested in immunostaining of sialyl glycan contaminenated/modified human bone marrow-derived mesenchymal stem cells (MSC) generated as described (Leskelä et al, 2003). The cell culture conditions with animal material (fetal calf serum, FCS) make the cells Neu5Gc and unusual oligosaccharide positive. Briefly, bone marrow obtained during orthopedic surgery was cultured in Minimum Essential alpha-Medium (α-MEM), supplemented with 20 mM HEPES, 10% FCS, penicillin-streptomycin and 2 mM L-glutamine (Gibco). After allowing to attach for 2 days, the cells were washed with PBS and subcultured at a density of 2000-3000 cells/cm² in the same medium. For immunostaining experiments, MSCs were cultured on coated glass 8-chamber slides and fixed with paraformaldehyde as described above for LLC-PK1 cells. Antibody Fab fragments were diluted in 1% HSA-PBS and incubated for 60 min at RT followed by washings 3 times 10 min with PBS. FITC-labelled goat anti-human lambda antibody (1:1000, Southern Biotechnology) was incubated for 60 min at RT, and washed 3 times for 5-10 min with PBS before mounting. The cells were observed with Zeiss Axioskop 2 plus fluorescence microscope (Carl Zeiss Vision GmbH, Germany) with fluorescein and DAPI filters. Images were taken with Zeiss AxioCam MRc camera and with AxioVision Software 3.1/4.0 (Carl Zeiss) with 400× magnification. Intensity of the stainings was graded as − (negative) or +/++/+++ (positive). Results are shown in Table 7. The antibodies 1.4.24 and 1.4.30, where found especially useful for recognizing the stem cells.

### Analysis of Sialic Acid Affecting Cell Culture Condition of Mesenchymal Stem Cells

Production of cord blood mesenchymal stem cells: Human term umbilical cord blood units were collected after delivery with informed consent of the mothers and the cord blood was processed within 24 hours of collection. Mononuclear cells (MNC:s) were isolated from each unit by Ficoll-Paque Plus (GE Healthcare Biosciences) density gradient centrifugation. The mononuclear cell fraction was plated on fibronectin (Sigma Aldrich)—coated 6-well plates (Nunc) at 10⁶ cells/well. Most of the non-adherent cells were removed as the medium was replaced the next day. The cells were cultured essentially as described for BM MSC:s above. The CB MSC:s used in the analyses were of passage 5-7.

Both BM and CB MSCs were analyzed by flow cytometry to be negative for CD14, CD34, CD45 and HLA-DR; and positive for CD13, CD29, CD44, CD90, CD105 and HLA-ABC. The cells were shown to be able to differentiate along osteogenic, adipogenic and chondrogenic lineages.

The cells were cultivated in presence of fetal cal serum. The cells cultivated in presence of FCS accumulated sialyl-oligosaccahride epitopes observable by 1.4.24 Fab fragment. A part of these could be removed by a neuraminidase treatment (not optimized) showing that the binding was sialic acid dependent. When cells are grown in presence of non-animal/unusual sialic acid glycan containing material (especially human serum), the antibody does not label the cells effectively. The invention is directed to the labelling of the stem cells and presence of the special sialic acid epitope, when correlated with culture in the presence of animal sialyl-material and not correlating with cultivation with xeno-free human material such as human serum.

### Example of an Antibody Variant

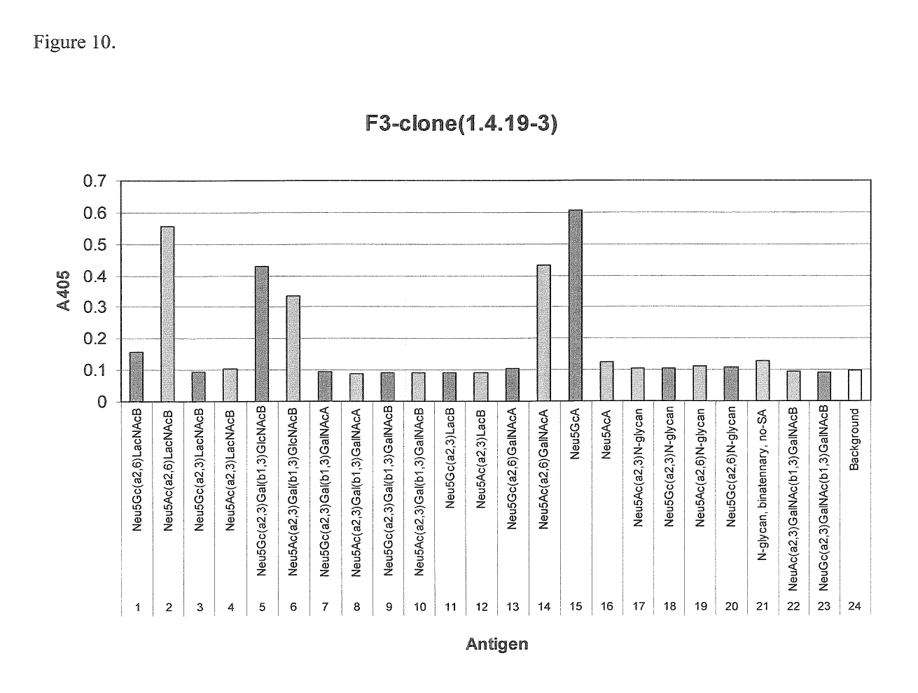

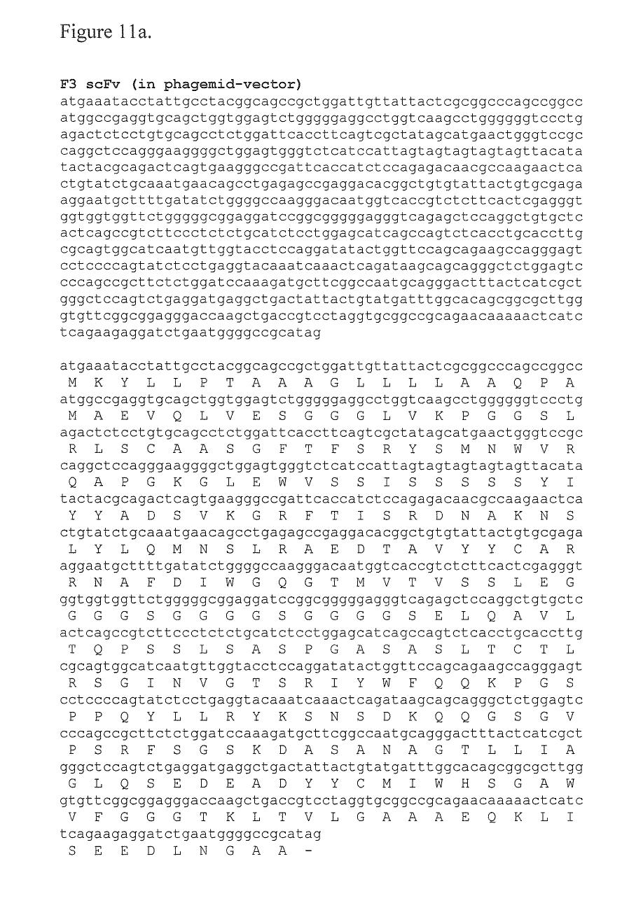

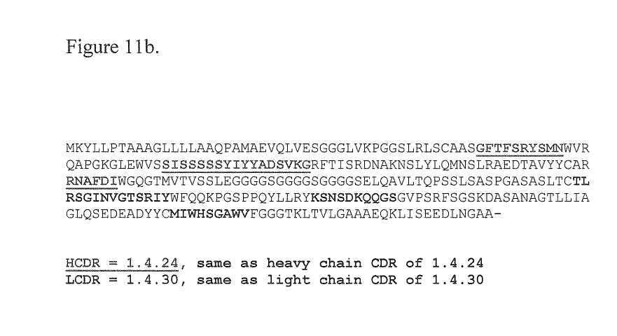

The screening of phage display library revealed a further antibody sequence referred as 1.4.19.(−3) also referred as F3. The sequence of the antibody includes heavy chain of 1.4.24 and light chain of 1.4.19, FIGS. 11a and 11b. The specificity and activity of the antibody is similar to 1.24.4 indicating that the heavy chain is a key factor determining the antibody specificity FIG. 10. The data further indicates that the light chains are at elats in part interchangeable. In a preferred embodiment the invention is directed to antibodies comprising the heavy sequences of antibody 1.4.24, with any of the four other antibodies, more preferably 1.4. group sequences, most preferably 1.4.24 or 1.4.30 (F3) light chain; or the heavy sequences of antibody 1.4.30, with any of the four other antibodies, more preferably 1.4. group sequences, most preferably 1.4.24 or 1.4.30 light chain;

TABLE 1
Binding of the selected NeuGc-binding antibody phage
clones to pig kidney tubular cells (LLC-PK1). The
binding was assessed by immunostaining and the specificity
by sialidase treatment of the cells.

Immunostaining intensity

| Phage antibody clone | untreated cells | sialidase treated |
|---|---|---|
| 1.2.20 | + | − |
| 1.4.11 | + | − |
| 1.4.24 | ++ | − |
| 1.4.30 | ++ | − |

TABLE 2
Certain background antibodies with similar protein sequences
especially similarity with 1.4.30 and possibly indicated
to bind specific glycolipids or other carbohydrates.

| | |
|---|---|
| WO 2006084050 | heparan sulfate, phosphorylated polypeptides |
| WO 2005094159 | Need to be checked if glycosylation is indicated. |
| WO 2002092017 | capsular polysaccharide (PPS-3). |
| WO 2002087611 | anti-GD2-antibodies, antiidiotypic antibodies against |
| | anti-GD2-antibodies |
| WO 2000073430 | Thomsen-Friedenreich (carbohydrate), |
| | Galbeta3GalNAcalfa, anti-MUC1 |
| U.S. Pat. No. | gangliosides GD3 and GQ1b |
| 5,730,981 | |
| AAO18444 protein | Anti-GD2 antibody |
| WO 2005005636 | GM2 and GM3 |
| ADD28053 protein | IgG glycosylation? |
| AEJ60702 protein | negatively charged carbohydrate or polypeptide |

TABLE 3
PCR primers library

Human VH back primers

| | |
|---|---|
| VH1a | 5′-GTCCTCGCAACTGCGGCCCAGCCGGCCATGGCCCAGGTGCAGCTGGTGCAGTCTGG-3′ |
| VH2a | 5′-GTCCTCGCAACTGCGGCCCAGCCGGCCATGGCCCAGGTCAACTTAAGGGAGTCTGG-3′ |
| VH3a | 5′-GTCCTCGCAACTGCGGCCCAGCCGGCCATGGCCGAGGTGCAGCTGGTGGAGTCTGG-3′ |
| VH4a | 5′-GTCCTCGCAACTGCGGCCCAGCCGGCCATGGCCCAGGTGCAGCTGCAGGAGTCGGG-3′ |
| VH5a | 5′-GTCCTCGCAACTGCGGCCCAGCCGGCCATGGCCCAGGTGCAGCTGTTGCAGTCTGC-3′ |
| VH6a | 5′-GTCCTCGCAACTGCGGCCCAGCCGGCCATGGCCCAGGTACAGCTGCAGCAGTCAGG-3′ |

Human JH forward primers

| | |
|---|---|
| JH1-2 | 5′-ATTTACTCGAGTGAGGAGACGGTGACCAGGGTGCC-3′ |
| JH3 | 5′-ATTTACTCGAGTGAAGAGACGGTGACCATTGTCCC-3′ |
| JH4-5 | 5′-ATTTACTCGAGTGAGGAGACGGTGACCAGGGTTCC-3′ |
| JH6 | 5′-ATTTACTCGAGTGAGGAGACGGTGACCGTGGTCCC-3′ |

Human Vκ back primers

| | |
|---|---|
| Vκ1a | 5′-TTATAGAGCTCGACATCCAGATGACCCAGTCTCC-3′ |
| Vκ2a | 5′-TTATAGAGCTCGATGTTGTGATGACTCAGTCTCC-3′ |
| Vκ3a | 5′-TTATAGAGCTCGAAATTGTGTTGACGCAGTCTCC-3′ |
| Vκ4a | 5′-TTATAGAGCTCGACATCGTGATGACCCAGTCTCC-3′ |
| Vκ5a | 5′-TTATAGAGCTCGAAACGACACTCACGCAGTCTCC-3′ |
| Vκ6a | 5′-TTATAGAGCTCGAAATTGTGCTGACTCAGTCTCC-3′ |

Human Jκ forward primers

| | |
|---|---|
| Jκ1 | 5′-TATAAGCGGCCGCACGTTTGATTTCCACCTTGGTCCC-3′ |
| Jκ2 | 5′-TATAAGCGGCCGCACGTTTGATCTCCAGCTTGGTCCC-3′ |
| Jκ3 | 5′-TATAAGCGGCCGCACGTTTGATATCCACTTTGGTCCC-3′ |
| Jκ4 | 5′-TATAAGCGGCCGCACGTTTGATCTCCACCTTGGTCCC-3′ |
| Jκ5 | 5′-TATAAGCGGCCGCACGTTTAATCTCCAGTCGTGTCCC-3′ |

Human Vλ back primers

| | |
|---|---|
| Vλ1 | 5′-ATTTAGAGCTCCAGTCTGTGTTGACGCAGCCGCC-3′ |
| Vλ2 | 5′-ATTTAGAGCTCCAGTCTGCCCTGACTCAGCCTGC-3′ |
| Vλ3a | 5′-ATTTAGAGCTCTCCTATGTGCTGACTCAGCCACC-3′ |
| Vλ3b | 5′-ATTTAGAGCTCTCTTCTGAGCTGACTCAGGACCC-3′ |
| Vλ4 | 5′-ATTTAGAGCTCCACGTTATACTGACTCAACCGCC-3′ |
| Vλ5 | 5′-ATTTAGAGCTCCAGGCTGTGCTCACTCAGCCGTC-3′ |
| Vλ6 | 5′-ATTTAGAGCTCAATTTTATGCTGACTCAGCCCCA-3′ |

Human Jλ forward primers

| | |
|---|---|
| Jλ1 | 5′-ATATTGCGGCCGCACCTAGGACGGTGACCTTGGTCCC-3′ |
| Jλ-3 | 5′-ATATTGCGGCCGCACCTAGGACGGTCAGCTTGGTCCC-3′ |
| Jλ4-5 | 5′-ATATTGCGGCCGCACCTAAAACGGTGAGCTGGGTCCC-3′ |

TABLE 4
Primers for amplification of Fab fragments

| | |
|---|---|
| Vλa5′ | 5′-ttgttattgctagctgcacaaccagcaatggcacacgttatactgactc-3′ |
| Vλb5′ | 5′-ttgttattgctagctgcacaaccagcaatggcacaggctgtgctcactc-3′ |
| Vλ3′ | 5′-gggggcggccttgggctgacctaggacggtsascttggtcc-3′ |
| Cλ5′ | 5′-cagcccaaggccgccccc-3′ |
| Cλ3′ | 5′-aggtagggcgcgccttatgaacattctgcaggggc |
| VH5′ | 5′-actcattaggcaccccaggc-3′ |
| VH3′ | 5′-tgaggagacggtgacc-3′ |
| CH5′ | 5′-ggtcaccgtctcctcagcctccaccaa-3′ |
| CH3′ | 5′-tttagtttatgcggccgcttaatggtgatgatgatggtgacaagatttgggctctgc-3′ |

TABLE 5
Search and comparison of similarities of antibody sequences.
Light Chain

| | CDR1 | CDR2 | CDR3 |
|---|---|---|---|
| 1.4 group | TLRSGINVGX₁X₂RIY | KSX₁SDKQQG | MIWHX₁X₂AX₃W |
| 1.4.11 | TLRSGINVG AY RIY | KSDSDKQQGS | MIWHSGA. .WV |
| 1.4.30 | TLRSGINVG TS RIY | KSNSDKQQGS | MIWHSGA. .WV |
| 1.4.24 | TLRSGINVG TY RIY | KSDSDKQQGS | MIWHNRA. VV |
| search | G T Y RIY | KSXSDKQQGS | MIWHXXAXV |
| BAC01851 | TLRSGINVG TY RIY | KSDSDKQQGS | MIWHSSA. .VV |
| BAC01849 | TLRSGINVG TY RIY | KSDSDKQQGS | MIWHSSA. .SV |
| Akahori et al | | | |
| AAH71725 | TLRSGINVG SY RIY | KSDSDKQQGS | MIWHSSA. .WV |
| genomic seq | | | |
| PNAS 99, 16899 | | | |
| search | | KSNSDKQ | |
| | was found not in | immunoglobulins | |
| 1.2.20. type | GGDNLGGKSVH | DDRDRPS | QVWDSGSESVV |
| search: | GGDNLGGKSVH | DDRDRPS | QVWDSGSESVV |
| CAC94245 | GGDDIGTKNVH | YDRDRPS | QVWDSSSEHVV |
| Brauninger Eur J Immunol | | | |
| CAC43034 | QGDSLRTYYVG | DDRDRPS | VSGQVSGRQLV |
| Hufton SE Provisorium | | | |

Heavy Chain

| | CDR1 | CDR2 | CDR3 |
|---|---|---|---|
| 1.4 group | X₁TFX₂X_{3Y}X₄MX₅ | X₁ISX₂SX₃X₄X₅X₆YYADSVKGX₁X₂X₃X₄X₅X₆X₇D | |
| 1.4.11 | ITFRKYAMN | AISNSGSDTYYADSVKG | RPKGGGMDV |
| 1.4.30 | FTFSSYAMS | AISGSGGSTYYADSVKG | MK..AGFDP |
| 1.4.24 | FTFSRYSMN | SISSSSSYIYYADSVKG | RN...AFDI |
| search | ITFRKYAMN: | | |
| AAA17943 | FTFNKYAMN | ISGSGASTYYADSVKG | LIFWDLVRGATFEN |
| J. Immun 151, 5290-300 | | | |
| search | FTFSSYAMS: | | |
| AAK57765 | FTFSSYAMS | ISDSGYSTYYADSVKG | LIAVAGPGGY |
| Br J Haematol 166, 662-6 | | | |
| Salcedo I et al | | | |
| search | FTFSRYSMN: | | |
| CAA78004 | FTFSRYSMN | ISDTFTTIYYADSVKG | STAVRGITFDY |
| Mortari, F | | | |
| AAL59365 | FTFSGYSMN | ISSSSSTIYYADSVKG | EALAGNFDY |
| Lieby P et al | | | |
| 1.2.20. type | GTVNSYYWS | RVYSSGTTNLNPSLKS | DY . . . GTDY |
| search | GTVNSYYWS | | |
| AAV40121 | ISSGSYYWS | RIYTSGSTNYNPSLKS | LYRLDAFDI |
| Kolar GR et al | | | |
| Blood 104, 2981-87 | | | |
| search | | RVYSSGTTNLNPSLKS | |
| | GSFSGYYWS | RVYTSGSTNYNPSLKS | DYVYNRKWTLYYGMDV |

DYGTDY CDR3 sequence was not found in immunoglobulins.

TABLE 6
Glycan-polyacrylamide (PAA)-biotin conjugates and
their codes used fors pecificity determination
for 1.4.24 and 1.4.30 antibodies

| Code | Glycan-PAA-biotin |
|---|---|
| GF271 | G1cAβ |
| GF272 | Glcβ |
| GF308 | Neu5Acα |
| GF309 | Neu5Gcα |
| GF336 | Glcα |
| GF337 | Fucα |
| GF338 | Ga1NAcβ |
| GF339 | Galβ |
| GF340 | GalNAcα |
| GF341 | GlcA |
| GF342 | GlcNAcβ |
| GF343 | Manα |
| GF344 | Manβ |
| GF348 | Neu5Acα6GalNAcα |
| GF347 | Neu5Gcα6GalNAcα |
| GF346 | Neu5Acα6LacNAcβ |
| GF345 | Neu5Gcα6LacNAcβ |
| GF459 | Neu5Acα3LacNAcβ |
| GF460 | Neu5Gcα3LacNAcβ |
| GF461 | Neu5Acα3Galβ3GlcNAcβ |
| GF462 | Neu5Gcα3Galβ3GlcNAcβ |
| GF465 | Neu5Acα3Galβ3GalNAcβ |
| GF466 | Neu5Gcα3Galβ3GalNAcβ |
| GF467 | Neu5Acα3Galβ3GalNAcα |
| GF468 | Neu5Gcα3Galβ3GalNAcα |
| GF463 | Neu5Acα3Lacβ |
| GF464 | Neu5Gcα3Lacβ |

TABLE 7
Binding of the selected Sialyl-oligosaccharide-specific
antibody Fab fragments to human mesenchymal stem cells
(MSC). The binding was assessed by immunostaining.

| Antibody Fab fragment | Immunostaining intensity |
|---|---|
| 1.2.20 | + |
| 1.4.11 | + |
| 1.4.24 | +++ |
| 1.4.30 | +++ |

### REFERENCES

Barbas III, C. F., Kang, A. S., Lerner, R. A., and Benkovic, S. J. (1991) Proc. Natl. Acad. Sci. U.S.A. 88, 7978-7982.
Desplancq, D., King, D. J., Lawson, A. D. G., and Mountain, A. (1994) Protein Eng. 7, 1027-1033.
Holliger, P., Prospero, T., and Winter, G. (1993) Proc. Natl. Acad. Sci. U.S.A. 90, 6444-6448.
Kabat, E. A., Wu, T. T., Reid-Miller, M., Perry, H. M., and Gottesman, K. S. (1991) Sequences of Proteins of Immunological Interest, 4th Ed., U.S. Dept. of Health and Human Services, Bethesda, Md.
McCafferty, J., Griffiths, A. D., Winter, G., and Chiswell, F. J. (1990) Nature 348, 552-554.

1.-59. (canceled)

60. A human monoclonal antibody that binds specifically to terminal non-reducing end oligosaccharide sequences:
1) α3-sialylated type 1 N-acetyllactosamine sequence SAα3Galβ3GlcNAc, wherein SA is Neu5Gc or Neu5Ac, said sequence being preferably Neu5Gcα3Galβ3GlcNAc, and
2) SAα6Gal(NAc)n, wherein SA is sialic acid, preferably being Neu5Gc or Neu5Ac and n is 0 or 1, and preferably does not bind to
3) SAα3Galβ4Glc(NAc)ₙ, wherein SA is Neu5Gc or Neu5Ac and n is 0 or 1.

61. The monoclonal antibody according to claim 60, wherein the terminal non-reducing end SAα6Gal(NAc)n comprising saccharide includes α6-sialylated type 2 N-acetyllactosamine sequence SAα6Galβ4GlcNAc, wherein SA is Neu5Gc or Neu5Ac, and sialylated non-reducing end terminal Neu5Acα6GalNAc-structures, preferably sialyl-Tn sequence Neu5Acα6GalNAcα.

62. The monoclonal antibody according to claim 60, wherein the terminal non-reducing end monosaccharide residues further include:
1) xenoantigenic non-reducing end single terminal NeuGcα-monosaccharide residue, but said antibody does not bind to non-reducing end single terminal NeuAcα-monosaccharide residue linked from reducing end to a polymer carrier, and does not bind to
2) oligosaccharide sequences according to SAα3Galβ4Glc(NAc)ₙ, wherein SA is Neu5Gc or Neu5Ac and n is 0 or 1.

63. The monoclonal antibody according to claim 60, wherein the antibody binds to both α3-sialylated type 1 N-acetyllactosamine sequences Neu5Gcα3Galβ3GlcNAc, and Neu5Acα3Galβ3GlcNAc, and
wherein the antibody binds to terminal non-reducing end epitopes sialyl-Tn sequences Neu5Acα6GalNAcα, and
wherein the antibody binds to both α6-sialylated type 2 N-acetyllactosamine including Neu5Acα6Galβ4GlcNAc, and Neu5Gcα6Galβ4GlcNAc, and
wherein the antibody binds to terminal non-reducing end epitopes Neu5Acα6Galβ4GlcNAc with higher affinity than Neu5Gca6Galβ4GlcNAc, and/or more effectively to Neu5Gca3Galβ3GlcNAc than Neu5Aca3Galβ3GlcNAc and/or not to Neu5Gca6GalNAcα.

64. The monoclonal antibody according to claim 60, wherein the antibody is selected from the group consisting of: (a) a whole immunoglobulin molecule; (b) an scFv; (c) a chimeric antibody; (d) a Fab fragment; (e) a Fab′ fragment; (f) an F(ab′)₂; (g) an Fv; and (h) a disulfide linked Fv: g) scFv fragment or the Fab fragment is from an antibody belonging to an IgM subclass.

65. The monoclonal antibody according to claim 60, wherein said antibody is comprised in a test kit comprising a suitable container for transport and storage, or is for use in immunodiagnostics or immunotherapy.

66. A method of binding an antibody or a cell binder reagent to non-reducing end glycan structures according to claim 60.

67. The method according to claim 66 for analyzing status of a human stem cell population involving a step of:
contacting the cells with a binder reagent that binds to terminal non-reducing end oligosaccharide sequences according to claim 60;
preferably for the analysis of an effect of exogenous materials and/cell culture conditions to the cells and the binder reagent being a monoclonal antibody.

68. The method according to claim 66, for analysis of disease associated or a cell binding antibody, preferably human antibody, wherein the method includes step of measuring the specificity of the antibody towards the sialylated oligosaccharide and monosaccharide sequences as defined in claim 60, preferably measuring specificity with regard to 3 oligosaccharides.

69. The method according to claim 66 for detecting carbohydrate epitope binding antibodies, the method comprising the steps of:
a) searching from available sequence data antibody sequences having essentially similar or same CDR1 or CDR2 sequences as described in FIG. 3, 4, or 11;
b) contacting an antibody found in step a) with sialyl saccharide library comprising saccharide sequences as described in claim 60; and
c) detecting if said antibody binds to any of said sequences or have the same binding specificity as the antibody according to claim 60.

70. The method according to claim 66 for a method of preparing a monoclonal antibody according to claim 60, comprising the step of:
synthetically producing at least a portion of said antibody or antibody derivative.

71. The method according to claim 66, for detecting acidic saccharide and/or NeuGc in a sample, comprising the steps of:
obtaining said sample, and
detecting the saccharide by contacting said sample with a monoclonal antibody as defined in claim 60.

72. The method according to claim 66 for analysing status of a human stem cell population involving a step of contacting the cells with said binder reagent, or for the analysis of an effect of exogenous materials and/cell culture conditions to the cells and the binder reagent being a monoclonal antibody.

73. The method according to claim 72, wherein the analysis is directed to surface expression of glycan structures on an intact cell population, or wherein the labelling by the antibody is associated with cell culture conditions in the presence of non-human exogenous material and/or lack of the labelling is associated with cell culture conditions in the presence of human equivalent material; or wherein the labelling is associated with presence of non-human or animal type glycan structures in said non-human exogenous materials and/or the lack of labelling is associated with presence of human type glycan structures in said human equivalent materials and optionally the labelling is associated with presence of animal serum proteins, preferably FCS, and/or the lack of labelling is associated with presence of equivalents of human serum proteins; or wherein the labelling is directed to major subpopulation of the intact cells, more preferably at least 15%, even more preferably at least 75% of the cells such as human blood derived mesenchymal stem cells, more preferably cord blood or bone marrow derived mesenchymal stem cells.

74. The antibody according to claim 60 obtainable by the method as defined in claim 66.

75. An isolated DNA molecule encoding the monoclonal antibody according to claim 60, and fragments of such DNA, which encode at least one antibody chain of said antibody.

76. The DNA according to claim 75 being a DNA contained in a host cell, preferably selected from the group: DNA contained in a host cell being capable of expressing a monoclonal antibody or a fragment or derivative thereof as defined in claim 60 or at least one antibody chain of said antibody; DNA contained in a host cell for a method of preparing a monoclonal antibody according to claim 60, comprising the steps of:
culturing a host cell containing DNA according to said claim capable of expressing at least one antibody chain, and
recovering said antibody.

77. The DNA according to claim 76 in a cell, wherein the DNA is in a phage or microbial cell which presents an antibody fragment selected from the group: (a) an scFv; (b) a Fab fragment; (c) a Fab′ fragment; (d) an F(ab′)2; (e) an Fv; and (f) a disulfide linked Fv as defined in claim 64 as a fusion protein with a surface protein.

78. The DNA according to claim 76 in a cell for a method of selecting an antibody according to claim 60, comprising the step of selecting said antibody from a display library of antibody fragments containing said phage or cell, and optionally further selecting from the display library of antibody fragments so that first antibodies that do not bind to a non-reducing end single terminal NeuAcα-conjugate are selected, and then antibodies that bind to non-reducing end single terminal NeuGcα-conjugate are selected from the remaining antibodies.

79. The analog of human monoclonal antibody according to claim 60 that binds to terminal non-reducing end oligosaccharide sequences:
1) α3-sialylated type 1 N-acetyllactosamine sequence SAα3Galβ3GlcNAc, wherein SA is Neu5Gc or Neu5Ac, said sequence being preferably Neu5Gca3Galβ3GlcNAc, and/or
2) α6-sialylated type 2 N-acetyllactosamine sequence SAα6Galβ4GlcNAc, wherein SA is Neu5Gc or Neu5Ac, and/or
3) sialylated non-reducing end terminal Neu5Acα6GalNAc-structures, preferably sialyl-Tn sequences Neu5Acα6GalNAcα, and/or
terminal non-reducing end monosaccharide residues:
4) xenoantigenic non-reducing end single terminal NeuGcα-monosaccharide residue, but does not bind to non-reducing end single terminal NeuAcα-monosaccharide residue,
and preferably does not bind to
5) oligosaccharide sequences according to SAα3Galβ4Glc(NAc)ₙ, wherein SA is Neu5Gc or Neu5Ac and n is 0 or 1.


SEQUENCE LISTING (22 sequences)

SEQ ID NO: 1 - protein, 108 aa, Homo sapiens
HVILTQPPSVSVAPGQTASIPCGGDNLGGKSVHWYRQRPGQAPVLVLYDDRDRPSGVPDRFSGSNFGATATLIIARVEAGDEADYHCQVWDSGSESVVFGGGTKVTVL

SEQ ID NO: 2 - protein, 115 aa, Homo sapiens
QAVLTQPSSLSASPGASASLTCTLRSGINVGAYRIYWFQQKPGSPPQYLLRYKSDSDKQQGSGVPSRFSGSKDASANAGTLLIAGLQSEDEADYYCMIWHSGAWVFGGGTKLTVL

SEQ ID NO: 3 - protein, 115 aa, Homo sapiens
QAVLTQPSSLSASPGASASLTCTLRSGINVGTSRIYWFQQKPGSPPQYLLRYKSNSDKQQGSGVPSRFSGSKDASANAGTLLIAGLQSEDEADYYCMIWHSGAWVFGGGTKLTVL

SEQ ID NO: 4 - protein, 115 aa, Homo sapiens
QAVLTQPSSLSASPGASASLTCTLRSGINVGTYRIYWYQQKPGSPPQYLLRYKSDSDKQQGSGVPSRFSGSKDASANAGILLISGLQSEDEADYYCMIWHNRAVVFGGGTKLTVL

SEQ ID NO: 5 - protein, 114 aa, Homo sapiens
QVQLQQSGPGLVKPSETLSLTCTVSGGTVNSYYWSWIRQSAGTGLEWIGRVYSSGTTNLNPSLKSRVTMSVDPPKNQFSLKLSSVTAADTAVYYCATDYGTDYWGQGTTVTVSS

SEQ ID NO: 6 - protein, 118 aa, Homo sapiens
QVNLRESGGGLVQPGGSLRLSCAASGITFRKYAMNWVRQAPGKGLDWVSAISNSGSDTYYADSVKGRFTISRDNSKNTLYLQMNSLGAEDTAVYYCTRRPKGGGMDVWGQGTLVTVSS

SEQ ID NO: 7 - protein, 116 aa, Homo sapiens
QVNLRESGGGLVQPGGSLRLSCAASGFTFSSYAMSWVRQAPGKGLEWVSAISGSGGSTYYADSVKGRFTISRDNSKNTLYLQMNSLRAEDTAVYYCAKMKAGFDPWGQGTTVTVSS

SEQ ID NO: 8 - protein, 115 aa, Homo sapiens
EVQLVESGGGLVKPGGSLRLSCAASGFTFSRYSMNWVRQAPGKGLEWVSSISSSSSYIYYADSVKGRFTISRDNAKNSLYLQMNSLRAEDTAVYYCARRNAFDIWGQGTMVTVSS

SEQ ID NO: 9 - protein, 14 aa, Homo sapiens
  MISC_FEATURE (10)..(10): Xaa is Ala or Thr
TLRSGINVGXXRIY

SEQ ID NO: 10 - protein, 10 aa, Homo sapiens
  MISC_FEATURE (3)..(3): Xaa is Asn or Asp
KSXSDKQQGS

SEQ ID NO: 11 - protein, 10 aa, Homo sapiens
  MISC_FEATURE (5)..(5): Xaa is Ser or Asn
MIWHXXAXWV

SEQ ID NO: 12 - protein, 11 aa, Homo sapiens
GGDNLGGKSVH

SEQ ID NO: 13 - protein, 7 aa, Homo sapiens
DDRDRPS

SEQ ID NO: 14 - protein, 11 aa, Homo sapiens
QVWDSGSESVV

SEQ ID NO: 15 - protein, 9 aa, Homo sapiens
GTVNSYYWS

SEQ ID NO: 16 - protein, 13 aa, Homo sapiens
RVYSSGTTNLNPS

SEQ ID NO: 17 - protein, 6 aa, Homo sapiens
DYGTDY

SEQ ID NO: 18 - protein, 5 aa, Homo sapiens
GFTFR

SEQ ID NO: 19 - protein, 5 aa, Homo sapiens
GFTFS

SEQ ID NO: 20 - protein, 5 aa, Homo sapiens
GITFR

SEQ ID NO: 21 - protein, 5 aa, Homo sapiens
GITFS

SEQ ID NO: 22 - protein, 5 aa, Homo sapiens
TLRSG